(19) United States
(12) Patent Application Publication
(10) Pub. No.: US 2024/0398320 A1
(43) Pub. Date: Dec. 5, 2024

(54) STABILIZING LINEAR FRACTIONAL-ORDER DYNAMICAL NETWORKS AND ITS IMPLICATIONS IN MITIGATING EPILEPSY

(71) Applicant: University of Southern California, Los Angeles, CA (US)

(72) Inventors: Paul Bogdan, Los Angeles, CA (US); Sergio Pequito, Uppsala (SE); Guilherme Ramos, Lisboa (PT); Emily Reed, Columbus, OH (US)

(21) Appl. No.: 18/677,575

(22) Filed: May 29, 2024

Related U.S. Application Data
(60) Provisional application No. 63/470,153, filed on May 31, 2023.

(51) Int. Cl. A61B 5/377

(52) U.S. Cl. CPC A61B 5/377

(57) ABSTRACT

A system includes one or more processors, coupled with memory, to generate, according to a model corresponding to a state of a network of neurons of a brain of a predetermined patient, a first matrix indicative of activity among a plurality of neurons of the brain during an event of the brain corresponding to epilepsy, modify, according to the model, one or more elements of the first matrix into a second matrix indicative of mitigation of the event for the plurality of neurons, and provide, to the patient to mitigate the event according to the second matrix, a therapeutic response having a physical property based on the first matrix and the second matrix.

### CROSS-REFERENCE TO RELATED PATENT APPLICATIONS

This application claims the benefit of priority under 35 U.S.C. § 119 to U.S. Provisional Patent Application Ser. No. 63/470,153, entitled “Stabilizing Linear Fractional-Order Dynamical Networks and Its Implications in Mitigating Epilepsy,” filed May 31, 2023, the contents of all such applications being hereby incorporated by reference in its their entirety and for all purposes as if completely and fully set forth herein.

### STATEMENT OF GOVERNMENT INTEREST

This invention was made with government support under the following grants: CPS/CNS-1453860, CCF-1837131, MCB-1936775, CNS-1932620, CMMI-1936624, and DGE-1842487 awarded by the National Science Foundation. The government has certain rights in the invention.

### SUMMARY

Epilepsy significantly inhibits the quality of life for its approximately 50 million patients worldwide and results in $16 billion dollars in annual expenditures in the United States alone to treat. Unfortunately, 15 million of those patients are unresponsive to medication, and surgery success rates are in the range of 30%-70% due to our limited understanding of how and where the disease originates. Seizures may develop or begin because of an instability in the brain. Further, the brain may be modeled as a complex dynamical system. Stability criteria are dependent on an assumed model of the system. The model of the system may represent the brain and neurons in a network of neurons as one or more matrices which can be modified to achieve stability.

This technical solution is directed at least to generating a brain neural model according to a plurality of matrices to indicate activity of a brain event corresponding to epilepsy, and modifying the matrix to indicate mitigation of the brain event corresponding to epilepsy. For example, the technical solution can execute a model as discussed herein to detect an epileptic event, devise a therapeutic action responsive to properties of the epileptic event for the individual, and instruct a system to provide the therapeutic response. Thus, a technical solution for detection and mitigation of brain event is provided. In addition, the technical solution can provide a technical improvement to determine a therapeutic response to mitigate an occurring epileptic event at an accuracy, personalization level, and responsiveness speed beyond the capability of manual processes to achieve.

A computer architecture may be used to perform computations to provide the technical solutions described herein. For example, a computer or computer system may contain one or more processors and a memory having one or more instructions thereon that cause the processors to execute the instructions. The computer system may generate the model of the brain that represents a complex dynamical system. Additionally, the computer architecture may generate a plurality of matrices to represent the model and/or brain system and perform one or more operations on the plurality of matrices to modify a representative unstable system to be a stable system. Additionally, the computer system may communicate with a therapeutic system configured to deliver therapy to the patient to treat epilepsy based on corresponding operations performed on the matrices that model system stability. The computer system may also communicate with one or more biomedical sensors configured to receive and/or collect data from the patient, such as brain wave data or neuron activity data.

The matrices generated by the computer system that model stability of the neural system and ultimately guide treatment of epilepsy may be more computationally efficient than existing or other solutions. For example, the stable system (e.g., system without epilepsy) may be modeled using one square matrix and a single vector of size n. Additionally, the modeled stable system may only be subject to n constraints. Because the network of neurons to be monitored may be large and/or complex. the approximate solutions provided by the matrices may be advantageous, for example, in reducing computation times. Additionally, display of the solution(s) may be graphically intuitive for providers treating patients based on the solution(s).

The use of matrices and matrix operations to model the neural system and subsequently treat epilepsy based on the matrices may improve epilepsy treatment. Specifically, in patients where medication is ineffective in treating epilepsy, treatment based on the modeled system may be less invasive and more accurate than existing non-medicinal options. Additionally, the systems and methods described herein may achieve greater responsiveness from patients compared to current treatments. When treating a patient, the change in parameters of the neural system may be achieved by, for example, target release of a drug, electrical stimulation, ultrasound neurostimulation, optogenetics, and/or regulation of glia astrocytes.

At least one aspect is directed to a system. The system can include one or more processors, coupled with memory. The system can generate, according to a model corresponding to a state of a network of neurons of a brain of a predetermined patient, a first matrix indicative of activity among a plurality of neurons of the brain during an event of the brain corresponding to epilepsy. The system can modify, according to the model, one or more elements of the first matrix into a second matrix indicative of mitigation of the event for the plurality of neurons. The system can provide, to the patient to mitigate the event according to the second matrix, a therapeutic response having a physical property based on the first matrix and the second matrix.

At least one aspect is directed to a method. The method can include generating, by a processor according to a model corresponding to a state of a network of neurons of a brain of a predetermined patient, a first matrix indicative of activity among a plurality of neurons of the brain during an event of the brain corresponding to epilepsy. The method can include modifying, by the processor according to the model, one or more elements of the first matrix into a second matrix indicative of mitigation of the event for the plurality of neurons. The method can include providing, by the processor to the patient to mitigate the event according to the second matrix, a therapeutic response having a physical property based on the first matrix and the second matrix.

At least one aspect is directed to a non-transitory computer readable medium can include one or more instructions stored thereon and executable by a processor. The processor can generate according to a model corresponding to a state of a network of neurons of a brain of a predetermined patient, a first matrix indicative of activity among a plurality of neurons of the brain during an event of the brain corresponding to epilepsy. The processor can modify, according to the model, one or more elements of the first matrix into a second matrix indicative of mitigation of the event for the plurality of neurons. The processor can provide, to the patient to mitigate the event according to the second matrix, a therapeutic response having a physical property based on the first matrix and the second matrix.

The present disclosure provides tractable, necessary, and sufficient conditions for the global asymptotic stability of discrete-time linear fractional-order systems. The systems and methods described herein may stabilize linear fractional-order dynamical networks. The systems and methods may be applied to real-world situations (e.g., patients) and provide an understanding concerning new treatments for epilepsy.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the disclosure can be combined with other aspects. Aspects can be implemented in any convenient form. For example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect. As used in the specification and in the claims, the singular form of ‘a,’ ‘an,’ and ‘the’ include plural referents unless the context clearly dictates otherwise.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting embodiments of the present disclosure are described by way of example with reference to the accompanying figures, which are schematic and are not intended to be drawn to scale. Unless indicated as representing the background art, the figures represent aspects of the disclosure. For purposes of clarity, not every component may be labeled in every drawing. In the drawings:

FIGS. 1A and 1B show plots of a spatial matrix and fractional-order exponents before a seizure event, according to some implementations;

FIGS. 2A and 2B show plots of eigenvalues of A₀=A−D (α, 1) using the system parameters before and during the seizure, according to some implementations;

FIGS. 3A, 3B, 3C, 3D, 3E, 3F, 3G, and 3H show plots of a spatial matrix and fractional-order exponents during the seizure, and their updated values along with their updated eigenvalues after solving PC and PC, according to some implementations;

FIGS. 4A, 4B, 4C, and 4D show plots of the updated spatial matrix and fractional-order exponents as well as their new eigenvalues after solving P₁^{g} and P₂^{g}, respectively, according to some implementations;

FIGS. 5A and 5B are block diagrams depicting embodiments of computing devices useful in connection with the methods and systems described herein;

FIG. 6 is a block diagram depicting an example computer architecture useful in performing the methods and systems described herein, according to some implementations;

FIG. 7 is a flow diagram depicting a method of detection and mitigation of brain events, according to some implementations; and

FIG. 8 is a flow diagram depicting a method of detection and mitigation of brain events, according to some implementations.

The details of various embodiments of the methods and systems are set forth in the accompanying drawings and the description below.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

Epilepsy affects approximately 50 million people worldwide. The recurrence of seizures can be mitigated through the use of medication for only about 70% of people. Non-medicinal treatments exist, such as surgery or nerve stimulation, which change the neuronal dynamics that underpin the seizure. However, these treatments may be invasive to the patient and could potentially cause long-term side effects. Epilepsy and/or the brain system as a whole may be modeled as a complex dynamical system. Epilepsy may develop as a result of an instability in the complex dynamical system due to a critical transition in the system. Such critical transitions may be associated with long-term memory dynamics. The long-term memory dynamics may be captured through or modeled by linear fractional-order systems. The present disclosure provide a method to stabilize these systems using linear matrix inequalities. The present techniques may be applied to epileptic patients for use in mitigating epilepsy.

The systems and methods described herein may include a computer architecture used to generate, based on a model of a network of neurons, a plurality of matrices that correspond to neural activity, specifically neural activity associated with epilepsy. As stated above, epilepsy may occur as a result of an instability in the complex dynamical system of the brain. Thus, a first matrix generated by the computer architecture may indicate instability in the neural system. Using various matrix operations and optimizations, the computer architecture may modify the first, unstable matrix to produce a second matrix that indicates stability in the neural system (e.g., no seizure activity). The modifications made to the first matrix to produce the second matrix indicative of a stable system may correspond to real-world treatments for epilepsy. For example, correcting the instability of the neural system modeled by the matrices may be performed by changing parameters of the DTLFOS (e.g., fractional-order exponents or the matrices).

Operations performed by a computer to generate the matrices that model stability of the neural system may be more computationally efficient than existing or other solutions. For example, the stable system (e.g., system without epilepsy) may be modeled by the computer using one (n×n) matrix and a single vector of size n. Additionally, the modeled stable system may only be subject to n constraints. Many sensors may be used to monitor brain activity in epileptic patients. As such, the network of neurons to be monitored may be large and/or complex. Therefore, the approximate solutions provided by the computer-generated matrices (e.g., one (n×n) matrix, one vector of size n, n constraints) may be advantageous. Additionally, display of the solution(s) may be graphically intuitive for providers treating patients based on the solution(s).

The use of a computer system to generate matrices and perform matrix operations to model the neural system and subsequently treat epilepsy based on the matrices may improve epilepsy treatment. Specifically, in patients where medication is ineffective in treating epilepsy, treatment based on the computer-modeled system may be less invasive and more accurate than existing non-medicinal options. For example, a computer system configured to generate and modify matrices to arrive at a modeled stable system may communicate with one or more sensors (e.g., sensors configured to receive brain data and/or neuron activity) and one or more therapeutic output devices. The computer system may determine and communicate, to a therapeutic output device, a therapeutic treatment for a patient's epilepsy or epileptic event(s) that corresponds to the matrix operations performed by the computer to generate a modified or updated matrix that indicates system stability (e.g., no epileptic event). The therapeutic output device may deliver the determined therapy to the patient. Additionally, the systems and methods described herein may achieve greater responsiveness from patients compared to current treatments. When treating a patient, the change in parameters of the neural system may be achieved by, for example, target release of a drug, electrical stimulation, ultrasound neurostimulation, optogenetics, and/or regulation of glia astrocytes.

The systems and methods described herein may model stability of discrete-time linear fractional-order systems. Utilizing properties of discrete-time linear fractional-order systems, linear matrix inequalities may be generated and utilized to stabilize fractional-order systems. Stability may be imposed on complex dynamical systems (e.g., the brain) to deliver effective treatments of epilepsy and other neurological diseases.

The systems and methods described herein provide discrete-time linear fractional-order systems (DTLFOS) to model neural activity, specifically in the context of epilepsy. The DTLFOS may be described by:

$${Δ}^{a} x [ k + 1 ] = A x [ k ] \qquad ( Eqn . 1 )$$

where x∈ [symbol] ⁿ denotes the state and Δ^{a} is the Grunwald-Letnikov discretization of the fractional derivative. The Grünwald-Letnikov discretization for any i-th state (1≤i≤n) can be expressed as Δ^{a}^{ᵢ}xᵢ[k]=Σⱼ₌₀^{k}ψ(aᵢ, j)xᵢ[k−j], where aᵢ∈ [symbol] is the fractional-order coefficient of the ith state and

$$ψ ( {a}_{i} , j ) = \frac{Γ ( j - {a}_{i} )}{Γ ( - {a}_{i} Γ ( j + 1 ) )} ,$$

with Γ(·) denoting the Gamma function. The tuple (A, a) may be used to represent the DTLFOS described in equation (1). The DTLFOS described in equation (1) may be an infinite-dimensional linear system. The fractional-order exponents may determine the weights on previous states used to compute the next state. The weights on which the previous states are used to compute subsequent states may decay according to, for example, a power law. For example, as the fractional-order exponents approach 0, the next state may depend less on states in the past. The system may become linear time-invariant (LTI) responsive to all fractional-order exponents being equal to 0. The DTLFOS may be modeled by one or more spatial matrices. The fractional-order exponents of the DTLFOS may also be graphically modeled. Additionally, eigenvalues corresponding to each spatial matrix may be determined and displayed, for example, on a unit circle.

Global asymptotic stability in the context of DTLFOS may be considered when stabilizing the matrices described herein. For example, a DTLFOS defined by equation (1) may be globally asymptotically stable when the following two conditions hold:
1) for every ε>0 there exists δ>0 such that if ∥x[0]∥<δ, then ∥x [k]∥<ε for all k≥0, and
2)

$${lim}_{k \to ∞} ‖ x [ k ] ‖ = 0$$

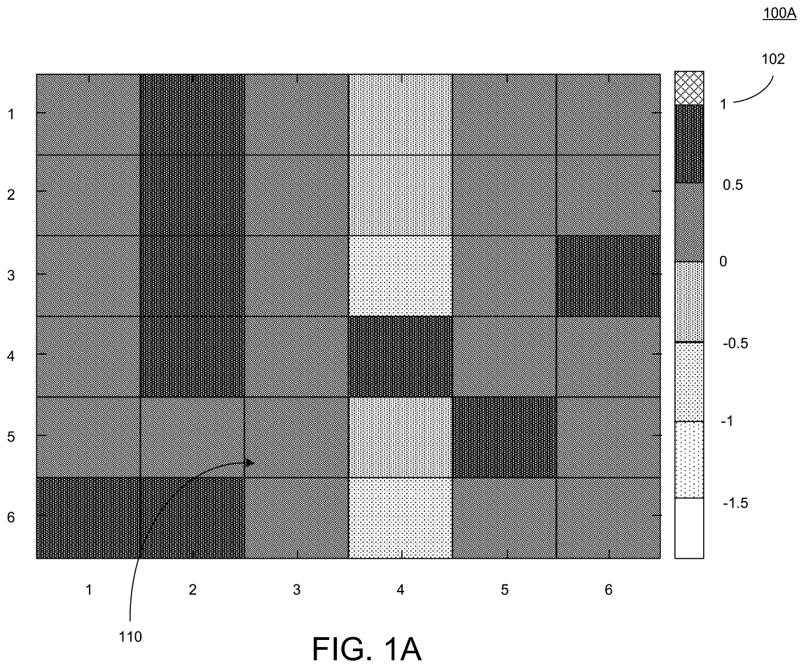

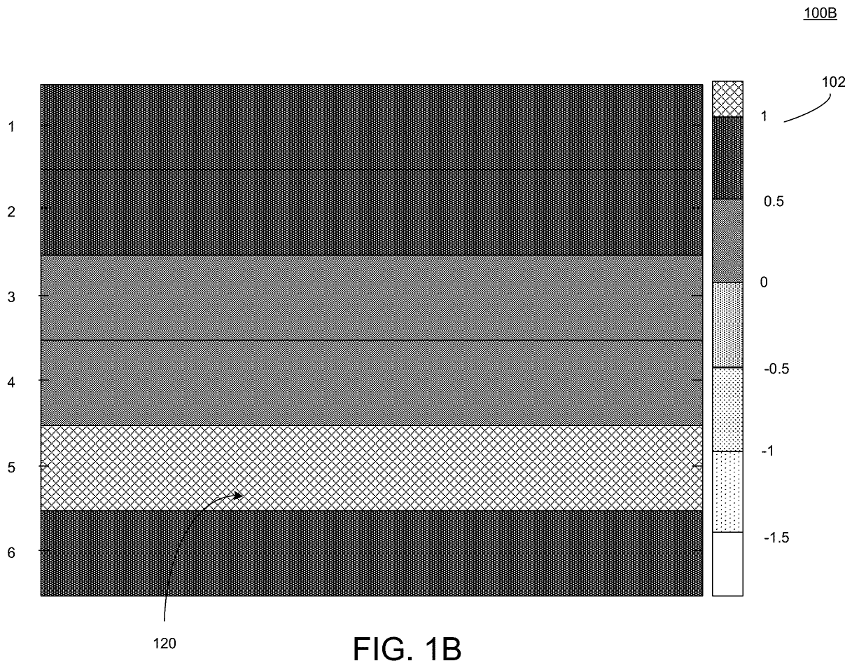

FIG. 1A depicts an example matrix model before seizure, according to this disclosure. As illustrated by way of example in FIG. 1A, a matrix model before seizure 100A can include at least a first model dimension 102, a second model dimension 104, and a matrix data 110. The tuple (A, a) described above may indicate a first state of a network of neurons. For example, the tuple (A, a) may indicate an unstable condition of a patient's neural system (e.g., a seizure is occurring or will occur). In various embodiments, the generation of the example matrix model of FIG. 1A may be executed by a computer system. In various embodiments still, the example matrix model may be generated by a processor configured for or optimized for matrix and/or multivariable calculations.

The first model dimension 102 can correspond to a number of rows in the matrix model before seizure 100A. For example, as shown in FIG. 1A, the first model dimension 102 is six. In various embodiments, the first model dimension 102 may be any number greater than zero. The first model dimension 102 may be represented with the letter n. The second model dimension 104 can correspond to a number of columns in the matrix model before seizure 100A. For example, as shown in FIG. 1A, the second model dimension 104 is six. In various embodiments, the second model dimension 104 may be any number greater than zero. The first model dimension 102 may be represented with the letter m. Dimensions of the matrix model before seizure 100A may be denoted as (n×m). In various embodiments, the first and second model dimensions 102, 104 may be equal to each other. Thus, dimensions of the matrix may be denoted as (n×n). As shown in FIG. 1A, the first model dimension 102 and the second model dimension 104 are equal (e.g., the matrix model before seizure 100A is a 6×6 matrix and the dimensions are shown as (6×6)). In various embodiments, the first and second model dimensions 102, 104 may be the same or different values. For example, the matrix model before seizure 100A may be a (4×7) matrix. The matrix data 110 can include a plurality of pieces of data. Each piece of data 110 may correspond to one (row, column) position within the matrix 110. For example, at each (row, column) position, the matrix may have a certain value. For example, as shown in FIG. 1A, value of the matrix at the (4, 1) position may be different than at the (3, 4) position.

FIG. 1B depicts an example exponent model before seizure, according to this disclosure. As illustrated by way of example in FIG. 1B, an exponent model before seizure 100B can include at least an exponent data 120. In various embodiments, the generation of the example exponent model of FIG. 1B may be executed by a computer system. In various embodiments still, the example exponent model may be generated by a processor configured for or optimized for matrix and/or multivariable calculations. The exponent data 120 can indicate a value of the fractional-order exponents for the DTLFOS. As stated above, fractional-order exponents may decay or approach zero, according to a power law. As shown in FIG. 1B, the exponent data 120 indicates that each fractional-order exponent has a value of around 0.75.

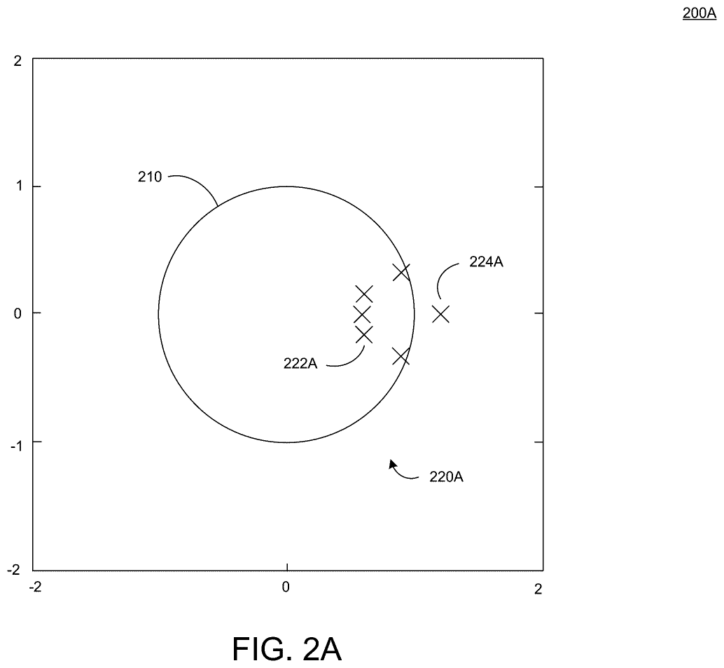

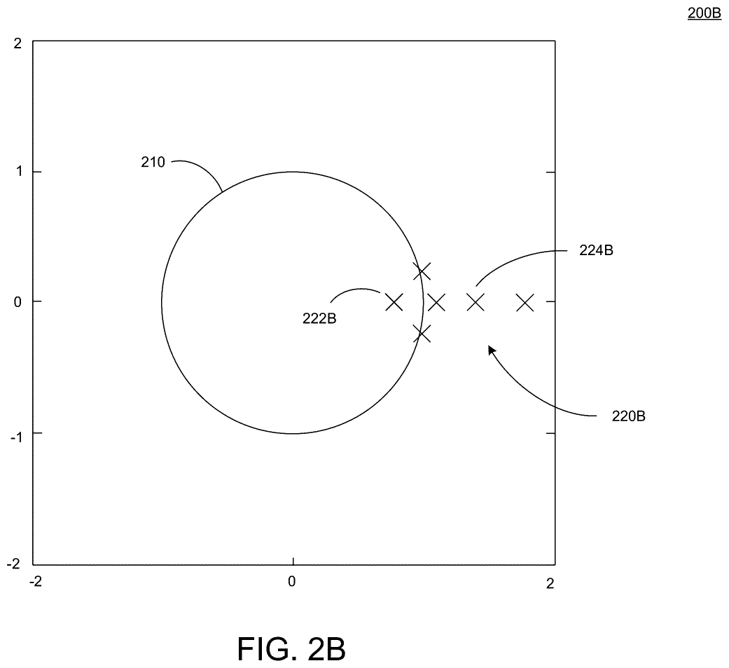

FIG. 2A depicts an example eigenvalue model before seizure, according to this disclosure. As illustrated by way of example in FIG. 2A, an eigenvalue model before seizure 200A can include at least a unit circle 210, and an eigenvalues for matrix model before seizure 220A. Specifically, FIG. 2A depicts eigenvalues of A₀=A−D (α, 1) using system parameters before seizure. The eigenvalues may be generated based on the example matrix model before seizure (e.g., matrix 100A). In various embodiments, the generation of the example eigenvalue model of FIG. 2A may be executed by a computer system. In various embodiments still, the example eigenvalue model may be generated by a processor configured for or optimized for matrix and/or multivariable calculations.

The unit circle 210 can indicate a state of stability of the system. For example, eigenvalues located within the unit circle 210 may indicate that the system is stable. Eigenvalues located on or outside of the unit circle 210 may indicate that the system is unstable. As stated above, the eigenvalues for matrix model before seizure 220A can lie within, on, and/or beyond the unit circle 210. The eigenvalues for matrix model before seizure 220A can include stable eigenvalues 222A, and an unstable eigenvalues 224A.

The stable eigenvalues 222A can be located within the unit circle 210. The stable eigenvalues 222A may indicate that the system is stable. However, the presence of stable eigenvalues 222A may not guarantee a stable system. For example, the presence of one or more unstable eigenvalues may indicate that the system is unstable. The unstable eigenvalues 224A can be located on or beyond the unit circle 210. An eigenvalue model depicting neural activity before seizure may indicate an unstable system. For example, as shown in FIG. 2A, the presence of unstable eigenvalues 224A, regardless of the presence of stable eigenvalues 222A, indicates that the system is unstable.

FIG. 2B depicts an example eigenvalue model during seizure, according to this disclosure. As illustrated by way of example in FIG. 2B, an eigenvalue model during seizure 200B can include at least an eigenvalues for matrix model during seizure 220B. Specifically, FIG. 2B depicts eigenvalues of A₀=A−D (α, 1) using system parameters during seizure. The eigenvalues may be generated based on the example matrix model during seizure (e.g., matrix 100B). In various embodiments, the generation of the example eigenvalue model of FIG. 2B may be executed by a computer system. In various embodiments still, the example eigenvalue model may be generated by a processor configured for or optimized for matrix and/or multivariable calculations.

The eigenvalues for matrix model during seizure 220B can lie within, on, and/or beyond the unit circle 210. As in FIG. 2A, the eigenvalues for matrix model during seizure 220B can include stable eigenvalues 222B, and an unstable eigenvalues 224B. The stable eigenvalues 222B can be located within the unit circle 210. Similar to FIG. 2A, the stable eigenvalues 222B may indicate that the system is stable. However, the presence of stable eigenvalues 222B may not guarantee a stable system. For example, the presence of one or more unstable eigenvalues may indicate that the system is unstable. The unstable eigenvalues 224B can be located on or beyond the unit circle 210. An eigenvalue model depicting neural activity during seizure may indicate an unstable system, because seizures may occur as a result of system instability due to critical transitions. For example, as shown in FIG. 2B, the presence of unstable eigenvalues 224A, even with the presence of stable eigenvalues 222A, indicate that a seizure is occurring.

In various embodiments, it may be determined that, both before and during a seizure, the system is unstable. For example, theorem 1, which will be described in greater detail below, may be utilized to determine the stability of the system. In various embodiments, the system before a seizure and the system during a seizure may possess different dynamical properties. For example, as shown by the eigenvalues 220A of FIG. 2A, the system is unstable as a result of the unstable eigenvalue 224A. As shown by the eigenvalues 220B of FIG. 2B, the system is unstable as a result of the unstable eigenvalues 224B. However, in FIG. 2A, there are a fewer number of unstable eigenvalues 224A, thus indicating, while both systems are unstable, each possesses different dynamical properties.

As stated above, while both systems before and during the seizure are unstable (e.g., as shown in FIGS. 2A and 2B), in the case of the system during the seizure (e.g., as shown in FIG. 2B), there are more eigenvalues outside of the unit circle (e.g., a greater value of unstable eigenvalues 224B. This may indicate that the system moves further from stability as a seizure begins and during the seizure. Thus, to mitigate the seizure, the system may be corrected for instability. As described herein, the systems and methods may correct for instability by, for example, altering the fractional-order exponents or the spatial matrix.

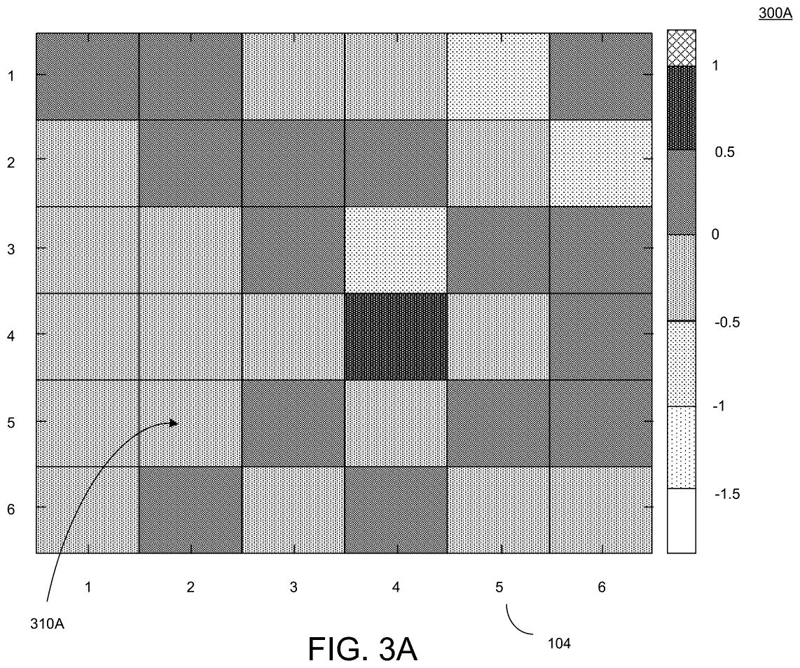

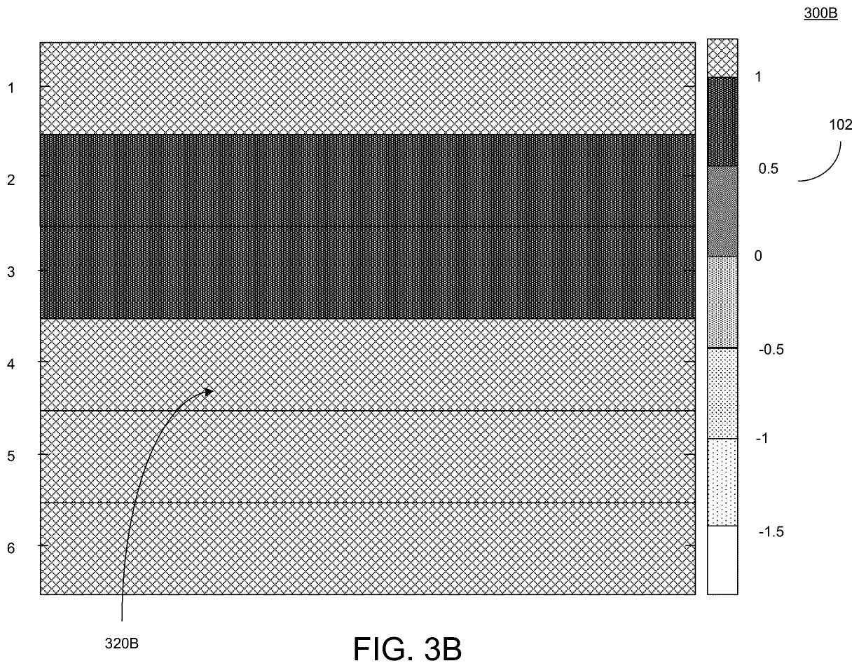

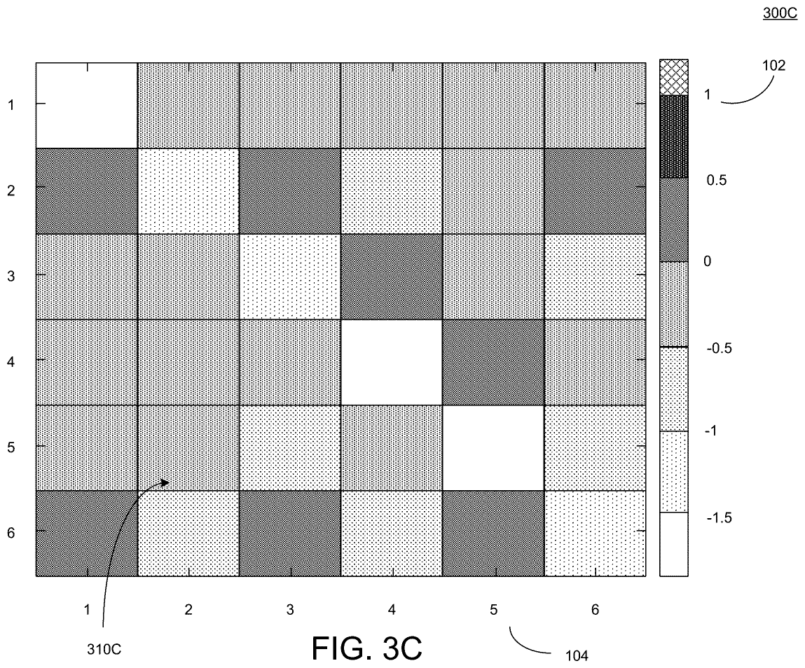

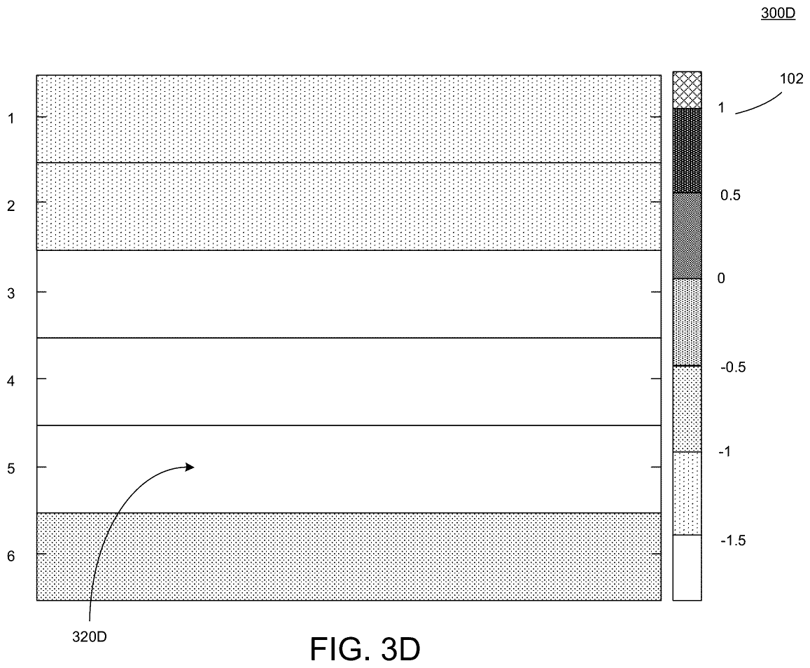

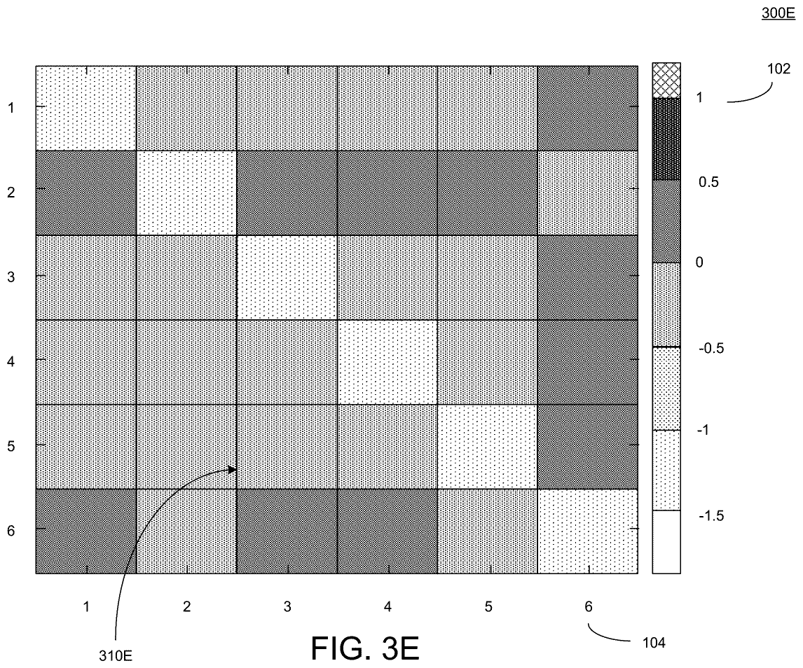

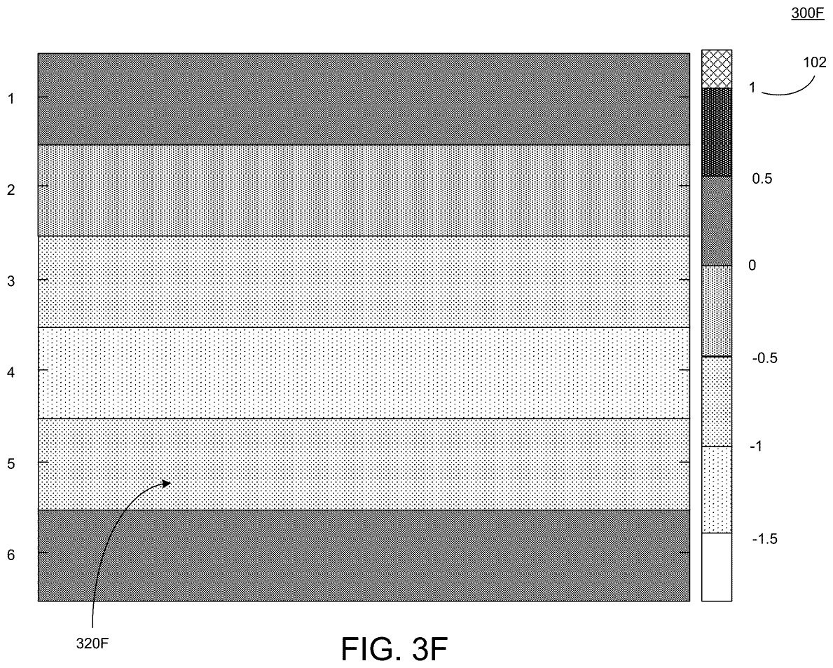

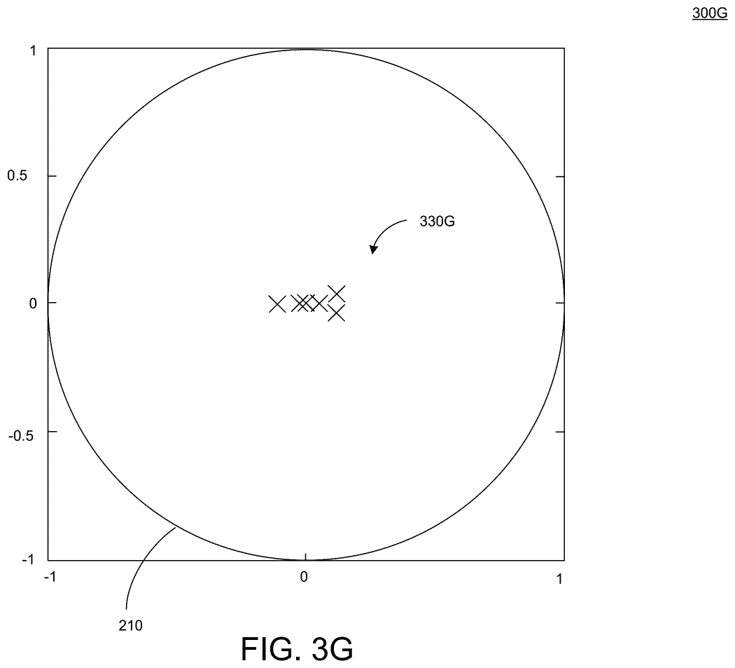

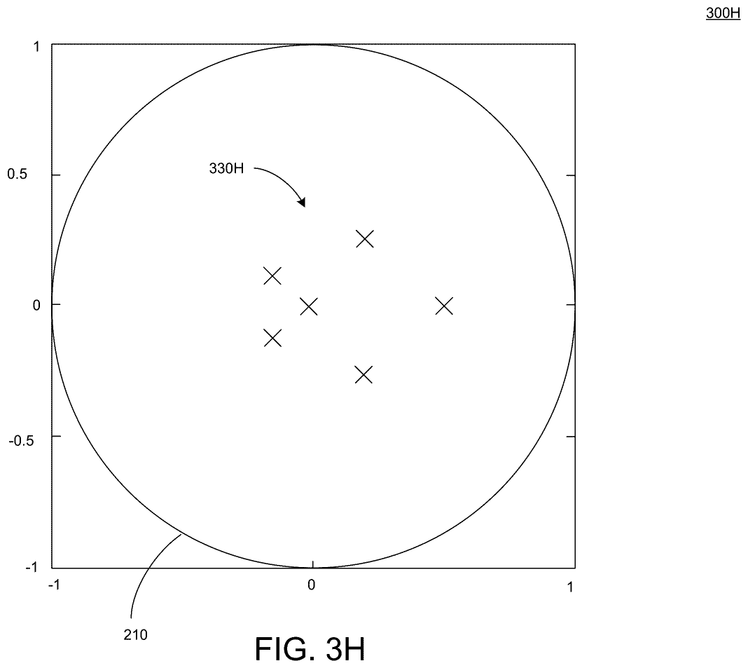

FIG. 3A depicts an example matrix model during seizure, according to this disclosure. As illustrated by way of example in FIG. 3A, a matrix model during seizure 300A can include at least a matrix data 310A. Specifically, FIG. 3A depicts an example spatial matrix A according to the DTLFOS. In various embodiments, the generation of the example matrix model of FIG. 3A may be executed by a computer system. In various embodiments still, the example matrix model may be generated by a processor configured for or optimized for matrix and/or multivariable calculations. The matrix data 310A, as a whole, can indicate neural activity. For example, in FIG. 3A, the (row, column) values as a whole may indicate that a seizure is occurring.

FIG. 3B depicts an example exponent model before seizure, according to this disclosure. As illustrated by way of example in FIG. 3B, an exponent model before seizure 300B can include at least an exponent data 320B. Specifically, FIG. 3B depicts example fractional order exponents a. In various embodiments, the generation of the example exponent model of FIG. 3B may be executed by a computer system. In various embodiments still, the example exponent model may be generated by a processor configured for or optimized for matrix and/or multivariable calculations. The exponent data 320B can, similar to the exponent data 120 of FIG. 1B, indicate a value of the fractional-order exponents for the DTLFOS. As shown in FIG. 3B, the exponent data 320 indicates that each fractional-order exponent has a value of around 1.

In various embodiments, a seizure may occur due to an instability caused by a critical transition in the complex dynamical system (e.g., the brain). Additionally, as shown in FIG. 2B, during a seizure, the system may be unstable. Thus, for neuron data recorded during a seizure, spatial matrices and corresponding fractional-order exponents may indicate instability. As such, stabilizing the brain network when modeled as a DTLFOS may be beneficial in treating seizures. To stabilize the system, interconnections between different states (e.g., current and previous states) may be altered or modified. Altering the interconnections may, in terms of generated spatial matrices, represent inter-dependencies among neuronal populations.

To alter or modify the interconnections between different states, the objective function may be represented as: given (A, α), find Ã that satisfies the following:

$${minimize}_{Ã ∈ {ℝ}^{n \times n}} {‖ Ã ‖}_{0} s . t . ( A + Ã , α ) is globally asymptotically stable , \qquad ( P1 )$$

where ∥·∥₀ represents a zero quasi-norm, which measures the number of non-zero entries in a matrix or vector. When α=0, the system may be a LTI system. When α≠0, altering the fractional-order exponents may achieve system stability. Thus, the following objective function can be determined: given (A, α), find ã that satisfies the following:

$${minimize}_{\tilde{a} ∈ {ℝ}^{n}} {‖ \tilde{a} ‖}_{0} s . t . ( A , α + \tilde{a} ) is globally asymptotically stable . \qquad ( P2 )$$

The objective functions P₁ and P₂ above indicate that it may be possible to change memory dependency of specific brain regions. Thus may indicate that a lack of asymptotic stability may result from, for example, too much or too little integration of the memory in a neural region.

FIG. 3C depicts an example mitigation matrix model for seizure, according to this disclosure. As illustrated by way of example in FIG. 3C, a mitigation matrix model for seizure 300C can include at least a matrix data 310C. Specifically, FIG. 3C depicts an altered or modified spatial matrix 300C that is generated when P1 is solved (e.g., ∥Ã∥₀ is minimized). In various embodiments, the generation of the example mitigation matrix model of FIG. 3C may be executed by a computer system. In various embodiments still, the example mitigation matrix model may be generated by a processor configured for or optimized for matrix and/or multivariable calculations. The matrix data 310C can indicate an altered or modified spatial matrix. For example, as shown in FIG. 3C, a diagonal of matrix data 310C having similar values generated upon alteration of the first matrix (e.g., matrix 100A). The values of the matrix data 310C may indicate stability or instability of the system.

FIG. 3D depicts an example mitigation exponent model for seizure, according to this disclosure. As illustrated by way of example in FIG. 3D, a mitigation exponent model for seizure 300D can include at least an exponent data 320D. Specifically, FIG. 3D represents altered or modified fractional order exponents (α+\tilde{α}) during a seizure that are determined when P2 is solved (e.g., ∥ã∥₀ is minimized). In various embodiments, the generation of the example mitigation exponent model of FIG. 3D may be executed by a computer system. In various embodiments still, the example mitigation exponent model may be generated by a processor configured for or optimized for matrix and/or multivariable calculations. The exponent data 320D can indicate a value of the fractional-order exponents during a seizure. For example, exponent data 320D indicates that each exponent has a value of around −1 to −1.5. This may indicate that the spatial matrix has been altered, as the exponent values have been altered relative to the exponent data 320B.

In various embodiments, global asymptotic stability of the system may allow proper treatment of seizures to be determined. Thus, convexification of the problems P1 and P2 to generate convexified solutions, may enable sufficient and computationally efficient solutions to P1 and P2. In various embodiments, the present disclosure may provide a closed-form solution to assess the global asymptotic stability of the DTLFOS. Theorem 1 below may affect the determination of stability of the system.

Theorem 1: A non-commensurate DTLFOS (Eqn. 1) is said to be globally asymptotically stable if and only if ∀λ∈σ(A₀). λ<1, where A₀: =A−D(α, 1),

$$∀ j > 0 , D ( α , j ) = [ \begin{matrix}ψ ( {α}_{1} , j ) & 0 … & 0 \\ 0 & ψ ( {α}_{2} , j ) … & 0 \\ 0 & ⋮ & ⋱ 0 \\ 0 & 0 & … ψ ( {α}_{n} , j )\end{matrix} ] ,$$

and σ(A₀) is the set of eigenvalues of matrix A₀.

Proof of Theorem 1: To present the global asymptotic stability conditions of the non-commensurate DTLFOS, the DTLFOS shown in (Eqn. 1) may be rewritten as:

$$x [ k + 1 ] = {\sum }_{j = 0}^{k} {A}_{j} x [ k - j ] , \\ where {A}_{0} = A - D ( α , 1 ) , {A}_{j} = - D ( α , j + 1 ) , for j \geq 1 , and \qquad ( Eqn . 4 )$$

$$D ( α , j ) = [ \begin{matrix}ψ ( {α}_{1} , j & 0 & … & 0 \\ 0 & ψ ( {α}_{2} , j ) & … & 0 \\ 0 & ⋮ & ⋱ & 0 \\ 0 & 0 & … & ψ ( {α}_{n} , j )\end{matrix} ] . \qquad ( Eqn . 5 )$$

Next, (Eqn. 1) may be written in infinite dimensions, which may produce the following:

$$[ \begin{matrix}x [ 1 ] \\ x [ 2 ] \\ x [ 3 ] \\ ⋮\end{matrix} ] = {\underbrace{[ \begin{matrix}{A}_{0} & 0 & 0 & 0 & … \\ {A}_{1} & {A}_{0} & 0 & 0 & … \\ {A}_{2} & {A}_{1} & {A}_{0} & 0 & … \\ ⋮ & ⋮ & ⋮ & ⋮ & ⋱\end{matrix} ]}}_{𝒜} [ \begin{matrix}x [ 0 ] \\ x [ 1 ] \\ x [ 2 ] \\ ⋮\end{matrix} ] \qquad ( Eqn . 6 )$$

The system in (Eqn. 6) may be an infinite-dimensional linear time-invariant system.

Additionally, the dimension of [symbol] is countably infinite since it is described by an infinite set of finite-dimensional matrices. Therefore, the spectrum of A is countably infinite. From the point spectrum of an operator T, denoted by spec (T), the following exists:

$$spec ( 𝒜 ) = spec ( [ \begin{matrix}{A}_{0} & 0 & 0 & 0 & … \\ {A}_{1} & {A}_{0} & 0 & 0 & … \\ {A}_{2} & {A}_{1} & {A}_{0} & 0 & … \\ ⋮ & ⋮ & ⋮ & ⋮ & ⋱\end{matrix} ] ) = spe c ( {A}_{0} ) ⋃ spec ( [ \begin{matrix}{A}_{0} & 0 & 0 & 0 & … \\ {A}_{1} & {A}_{0} & 0 & 0 & … \\ {A}_{2} & {A}_{1} & {A}_{0} & 0 & … \\ ⋮ & ⋮ & ⋮ & ⋮ & ⋱\end{matrix} ] ) ,$$

where the second equality is a consequence of the properties of matrix determinants and the Leibniz expansion. It follows that

$$spec ( 𝒜 ) = {⋃}^{∞} spec ( {A}_{0} ) \qquad ( Eqn . 7 )$$

where the symbol ∞ indicates the union of a countable collection of sets. Subsequently, it follows that

$$spec ( 𝒜 ) = spec ( {A}_{0} ) . \qquad ( Eqn . 8 )$$

Therefore, the stability conditions of linear time-invariant dynamical systems can be leveraged.

By utilizing Theorem 1, P1 and P2 may be written as:

$$minimize {‖ Ã ‖}_{0} with the constraint Ã ∈ {ℝ}^{n \times n} s . t . ρ ( A + Ã - D ( a , 1 ) ) < 1 \qquad ( P1 )$$

$$and$$

$$minimize {‖ \tilde{a} ‖}_{0} with \tilde{a} ∈ {ℝ}^{n} \\ s . t . ρ ( A - D ( a + \tilde{a} , 1 ) ) < 1 \qquad ( P2 )$$

where ρ(M)=max{|λ|: λ∈σ(M)} is the spectral radius, which may be the largest eigenvalue in magnitude of arbitrary matrix M∈ [symbol] ^{n×n}.

In various embodiments, the objective functions of P1 and P2 may be nonconvex. Thus by considering the sparsity promoting 1−norm, convexification of the objective functions may occur. Specifically, for the objective functions of P1 and P2, convexified versions of the objective functions may be expressed as:

$$minimize {‖ Ã ‖}_{1} with Ã ∈ {ℝ}^{n \times n} \\ s . t . ρ ( A + Ã - D ( a , 1 ) ) < 1 \qquad ( P1C )$$

$$and$$

$$minimize {‖ \tilde{a} ‖}_{1} with \tilde{a} ∈ {ℝ}^{n} \\ s . t . ρ ( A - D ( a + ã , 1 ) ) < 1 \qquad ( P2C )$$

The solution to P₁^{c} may be given by Proposition 1 below:

$$Ã = {L}_{1} {P}_{1}^{- 1} , \qquad ( Eqn . 2 )$$

where P1 and L1 are found by solving the following convex optimization problem:

$$minimize {‖ {P}_{1} ‖}_{1} + {‖ {L}_{1} ‖}_{1} with {P}_{1} ∈ \{ {P}_{1} ∈ {ℝ}^{n \times n} : {P}_{1} > 0 \} , {L}_{1} ∈ {ℝ}^{n \times n} \\ s . t . [ \begin{matrix}{P}_{1} & {P}_{1} {A}_{0}^{T} + {L}_{1}^{T} \\ {A}_{0} {P}_{1} + {L}_{1} & {P}_{1}\end{matrix} ] > 0$$

Proof of Proposition 1: To solve P1C, P1C can be restated as

$$minimize {‖ Ã ‖}_{1} with {P}_{1} ∈ \{ {P}_{1} ∈ {ℝ}^{n \times n} : {P}_{1} > 0 \} , Ã ∈ {ℝ}^{n \times n} \\ s . t . {( {A}_{0} + Ã )}^{T} {P}_{1} ( {A}_{0} + Ã ) - {P}_{1} < 0. \qquad ( P1 )$$

The problem then becomes:

$$minimize {‖ {P}_{1} ‖}_{1} + {‖ {L}_{1} ‖}_{1} with {P}_{1} ∈ \{ {P}_{1} ∈ {ℝ}^{n \times n} : {P}_{1} > 0 \} , {L}_{1} ∈ {ℝ}^{n \times n} \\ s . t . [ \begin{matrix}{P}_{1} & {P}_{1} {A}_{0}^{T} + {L}_{1}^{T} \\ {A}_{0} {P}_{1} + {L}_{1} & {P}_{1}\end{matrix} ] > 0 , \qquad ( {P}_{1}^{c} )$$

where Ã=L₁P₁⁻¹. Since P1C is convex, it can be solved for L1 and P1 by using the interior points method.

The solution to P2C may be given by Proposition 2 below:

A suboptimal solution to P2C may be expressed as:

$${ã}_{i} = Γ ( 2 ) ( {L}_{2} {P}_{2}^{- 1} ) i , i \qquad ( Eqn . 3 )$$

for all i∈{1, . . . , n}, where P2 and L2 are found by solving the convex optimization problem below:

$$\begin{matrix}minimize {‖ {P}_{2} ‖}_{1} + {‖ {L}_{2} ‖}_{1} with {P}_{2} ∈ \{ {P}_{2} ∈ {ℝ}^{n \times n} : {P}_{2} > 0 \} , {L}_{2} ∈ {ℝ}^{n \times n} \\ s . t . [ \begin{matrix}{P}_{2} & {P}_{2} {A}^{T} + {L}_{2}^{T} \\ {AP}_{2} + {L}_{2} & {P}_{2}\end{matrix} ] > 0 \\ {P}_{2} , {L}_{2} diagonal\end{matrix}$$

Proof of Proposition 2: Similar to Proposition 1, P2C is solved. P2C can be restated as:

$$\begin{matrix}minimize {‖ \tilde{α} ‖}_{1} with {P}_{2} ∈ \{ {P}_{2} ∈ {ℝ}^{n \times n} : {P}_{2} > 0 \} , \tilde{α} ∈ {ℝ}^{n} \\ s . t . {( A - D ( α + \tilde{α} , 1 ) )}^{T} {P}_{2} ( A - D ( α + \tilde{α} , 1 ) ) - {P}_{2} < 0 .\end{matrix} \qquad ( {P}_{2}^{c} )$$

Then, the following is obtained using an appropriate theorem:

$$\begin{matrix}minimize {‖ {P}_{2} ‖}_{1} + {‖ {L}_{2} ‖}_{1} with {P}_{2} ∈ \{ {P}_{2} ∈ {ℝ}^{n \times n} : {P}_{2} > 0 \} , {L}_{2} ∈ {ℝ}^{n \times n} \\ s . t . [ \begin{matrix}{P}_{2} & {P}_{2} {A}^{T} + {L}_{2}^{T} \\ A {P}_{2} + {L}_{2} & {P}_{2}\end{matrix} ] > 0\end{matrix} \qquad ( {P}_{2}^{c} )$$

where D(α+\tilde{α}, 1)=−L₁P₁⁻¹. Since D(α+\tilde{α}, 1) is diagonal, L2 and P2 are restricted to be diagonal, which imposes 2 (n²−n) additional linear constraints. P2C is convex and can be solved for L2 and P2 by using the interior points method. From D(α+\tilde{α}, 1)=−L₁P₁⁻¹, \tilde{α} may need to be obtained.

From equation (5), D(α+\tilde{α}, 1) is dependent on ψ(aᵢ+ãᵢ, 1) for all i∈{1, . . . , n} i.e.,

$$ψ ( {α}_{i} + {\tilde{α}}_{i} , 1 ) = \frac{Γ ( 1 - ( {a}_{i} + {\tilde{a}}_{i} ) )}{Γ ( - ( {a}_{i} + {\tilde{a}}_{i} ) ) Γ ( 2 )} . \qquad ( Eqn . 9 )$$

However, from the relationship Γ(1+z)=zΓ(z), ψ(αᵢ+\tilde{α}ᵢ, 1) can be simplified to

$$\frac{Γ ( 1 - ( {α}_{i} + {\tilde{α}}_{i} ) )}{Γ ( - ( {α}_{i} + {\tilde{α}}_{i} ) ) Γ ( 2 )} = \frac{- ( {α}_{i} + {\tilde{α}}_{i} )}{Γ ( 2 )} \frac{}{} \qquad ( Eqn . 10 )$$

Thus, D(α+\tilde{α}, 1) becomes

$$D ( α + \tilde{a} , 1 ) = [ \begin{matrix}\frac{- ( {α}_{1} + {\tilde{α}}_{1} )}{Γ ( 2 )} & 0 & … & 0 \\ 0 & \frac{- ( {α}_{2} + {\tilde{α}}_{2} )}{Γ ( 2 )} & … & 0 \\ 0 & ⋮ & ⋱ & \\ 0 & 0 & … & \frac{- ( {α}_{n} + {\tilde{α}}_{n} )}{Γ ( 2 )}\end{matrix} ] . \qquad ( Eqn . 11 )$$

By equating the diagonal entries of L₂P₂⁻¹ to the diagonal entries of D(α+\tilde{α}, 1), \tilde{α} is solved for and a result is obtained.

In various embodiments, in the solution to P2C elements of \tilde{α} may be altered to be possibly nonzero. Thus, \tilde{α} may correspond to diagonal entries of L₂P₂⁻¹. In various embodiments, \tilde{α} corresponding to diagonal entries of L₂P₂⁻¹ may be possible when both matrices L2 and P2 are diagonal and P2 is positive definite.

The systems and methods described herein may be or utilize an event-triggered state-feedback control. For example, the systems and methods may be governed by an equation u[k]=Kx[k], where u[k] is the control input. The systems and methods may be utilized or employed responsive to detection of a seizure. For example, in the case of P1C, K may be set to equal Ã. For P2C diagonal entries of

$${K}_{i , i} = \frac{- {\tilde{α}}_{i}}{Γ ( 2 )}$$

may be set for all i∈{1, . . . , n}, and off-diagonal entries may be zero.

FIG. 3E depicts an example second mitigation matrix model for seizure, according to this disclosure. As illustrated by way of example in FIG. 3E, a second mitigation matrix model for seizure 300E can include at least a matrix data 310E. Specifically, the matrix 300E may be a spatial matrix (A+Ã) corresponding to the solution to P1C. The matrix 300E may indicate a stable system. For example, the matrix 300E may be a matrix that results from the performance of matrix operations to stabilize a model of the neural system of a patient. In various embodiments, the generation of the example second mitigation matrix model of FIG. 3E may be executed by a computer system. In various embodiments still, the example second mitigation matrix model may be generated by a processor configured for or optimized for matrix and/or multivariable calculations. The matrix data 310E can indicate a stable system. As shown in FIG. 3E, across a diagonal, each matrix position has a value of around −1. Elsewhere, each matrix position has a value of around zero. This may indicate stability in the system. For example, the matrix 300A has been modified to now indicate a stable system.

FIG. 3F depicts an example second mitigation exponent model for seizure, according to this disclosure. As illustrated by way of example in FIG. 3F, a second mitigation exponent model for seizure 300F can include at least an exponent data 320F. Specifically, the matrix 300E may be a spatial matrix (α+ã) corresponding to the solution to P2C. In various embodiments, the generation of the example second mitigation exponent model of FIG. 3F may be executed by a computer system. In various embodiments still, the example second mitigation exponent model may be generated by a processor configured for or optimized for matrix and/or multivariable calculations. The exponent data 320F can indicate that the weight of the fractional-order exponents approach zero. This may indicate long term memory, and the fractional-order exponents may represent neural behavior accurately.

FIG. 3G depicts an example first mitigation eigenvalue model for seizure, according to this disclosure. As illustrated by way of example in FIG. 3G, a first mitigation eigenvalue model for seizure 300G can include at least an eigenvalues for mitigation matrix model 330G. Specifically, the first mitigation eigenvalue model for seizure 300G may be generated using the solution from P1C. In various embodiments, the generation of the example second mitigation eigenvalue model of FIG. 3G may be executed by a computer system. In various embodiments still, the example second mitigation eigenvalue model may be generated by a processor configured for or optimized for matrix and/or multivariable calculations. As shown in FIG. 3G, the eigenvalues for mitigation matrix model 330G all lie within the unit circle, indicating system stability. FIG. 3H depicts an example second mitigation eigenvalue model for seizure, according to this disclosure. As illustrated by way of example in FIG. 3H, a second mitigation eigenvalue model for seizure 300H can include at least an eigenvalues for mitigation matrix model 330H. Specifically, the first mitigation eigenvalue model for seizure 300H may be generated using the solution from P2C. In various embodiments, the generation of the example second mitigation eigenvalue model of FIG. 3H may be executed by a computer system. In various embodiments still, the example second mitigation eigenvalue model may be generated by a processor configured for or optimized for matrix and/or multivariable calculations. As shown in FIG. 3H, the eigenvalues for mitigation matrix model 330H all lie within the unit circle, indicating system stability.

In various embodiments, upon solving P1C, the updated or modified spatial matrix (A+Ã) (e.g., matrix 300E) may have lower values along a diagonal, as shown in FIG. 3E, compared to the original and altered or modified spatial matrices 300A and 300C, shown in FIGS. 3A and 3C, respectively. In various embodiments, the spatial matrix (e.g., matrix 300E) may indicate a level of activity between neuron populations. Thus, a spatial matrix having lower values may indicate decreased activity between neuron populations.

In various embodiments, after solving P2C, the updated or modified fractional-order exponents (α+ã) (e.g., exponent data 320F) shown in FIG. 3F may have lower values than the original and altered or modified fractional-order exponents (e.g., exponent data 320B and 320D) shown in FIGS. 3B and 3D, respectively. The original fractional-order exponents (a) (e.g., exponent data 320B) during the seizure may be close to 1. However, the updated or modified fractional-order exponents (e.g., exponent data 320F) may have lower values. For example, as shown in FIG. 3F, the updated fractional-order exponents may have values in the range of (−0.6, 0.5).

After solving P1C and P2C, updated or modified eigenvalues 330G and 330H for the new systems may be determined, as shown in FIGS. 3G and 3H. By invoking Theorem 1, the updated systems may be globally asymptotically stable.

In various embodiments, graphically-interpretable sufficient approximate solutions may be determined to solve P1C and P2C. This may allow the data shown in the updated or modified spatial matrices to be more easily viewed. For example, physicians or other care providers may view the approximate solutions and be able to easily visualize the data to determine appropriate epilepsy treatment. Proposition 3 below indicates a graphically-interpretable sufficient approximate solution for P1C.

The following problem formulation is sufficient for solving P1C:

$$minimize {‖ \tilde{A} ‖}_{1} with \tilde{A} ∈ {ℝ}^{n \times n} s . t . \vert {a}_{i , i} + {ã}_{i , i} + \frac{{α}_{i}}{Γ ( 2 )} \vert < 1 - {\sum }_{j ∈ N ∖ \{ i \}} \vert {a}_{i , j} + {ã}_{i , j} \vert . \qquad ( PG1 )$$

### Proof of Proposition 3:

Gershgorin's theorem and the reverse triangle inequality may be invoked. These may combine to say that ∀λ₁∈σ(A+Ã−D(α, 1))) there exists a positive integer i∈{1, . . . , n} such that the following holds

$$\vert {1}_{} \vert \leq \vert {a}_{i , i} + {\tilde{a}}_{i , i} - ψ ( {α}_{i} , 1 ) \vert + {\sum }_{j ∈ N ∖ \{ i \}} \vert {a}_{i , j} + {\tilde{a}}_{i , j} \vert . \qquad ( Eqn . 12 )$$

Thus, in providing sufficient graphical conditions to solve P1C, all ã_{i,j}, ∀i∈{1, . . . , n} and ∀i∈{1, . . . , n} may be sought such that

$$\vert {a}_{i , i} + {\tilde{a}}_{i , i} - ψ ( {α}_{i} , 1 ) \vert + {\sum }_{j ∈ N ∖ \{ i \}} \vert {a}_{i , j} + {\tilde{a}}_{i , j} \vert < 1. \qquad ( Eqn . 13 )$$

This implies the following:

$$\vert {a}_{i , i} + {\tilde{a}}_{i , i} - \frac{Γ ( 1 - {α}_{i} )}{Γ ( - {α}_{i} ) Γ ( 2 )} \vert < 1 - {\sum }_{j ∈ N ∖ \{ i \}} \vert {a}_{i , j} + {\tilde{a}}_{i , j} \vert . \qquad ( Eqn . 14 )$$

The sufficient graphical constraint in (Eqn. 14) implicitly requires that 1−Σ_{j∈N\{i}}|a_{i,j}+ã_{i,j}|>0, which indicates that the problem may not always be feasible. By the same relationship in equation (Eqn. 10), the result is obtained.

Similarly, proposition 4 below indicates a graphically-interpretable sufficient approximate solution for P2C.

The following problem formulation is sufficient for solving P2C:

$$minimize {‖ \tilde{α} ‖}_{1} with \tilde{α} ∈ {ℝ}^{n} \\ s . t . \vert {a}_{i , i} + \frac{{α}_{i} + {\tilde{α}}_{i}}{Γ ( 2 )} \vert < 1 - {\sum }_{j ∈ N ∖ \{ i \}} \vert {a}_{i , j} \vert . \qquad ( PG2 )$$

### Proof of Proposition 4:

Similar to the proof of Proposition 3, Gershgorin's theorem and the reverse triangle inequality are invoked, which combine to say that ∀λ₂∈σ(A−D (α+\tilde{α}, 1)) there exists a positive integer i∈{1, . . . , n} such that the following holds

$$\vert {2}_{} \vert \leq \vert {a}_{i , i} - ψ ( {α}_{i} + {\tilde{α}}_{i} , 1 ) \vert + {\sum }_{j ∈ N ∖ \{ i \}} \vert {a}_{i , j} \vert \qquad ( Eqn . 15 )$$

Thus, in providing sufficient graphical conditions to solve P2C, ãᵢ, ∀i∈{1, . . . , n} and ∀i∈{1, . . . , n} are sought such that

$$\vert {a}_{i , i} - ψ ( {α}_{i} + {\tilde{α}}_{i} , 1 ) \vert + {\sum }_{j ∈ N ∖ \{ i \}} \vert {a}_{i , j} \vert < 1 , \qquad ( Eqn . 16 )$$

which implies the following

$$\vert {a}_{i , i} - \frac{Γ ( 1 - ( {α}_{i} + {\tilde{α}}_{i} ) )}{Γ ( - ( {α}_{i} + {\tilde{α}}_{i} ) ) Γ ( 2 )} \vert < 1 - {\sum }_{j ∈ N ∖ \{ i \}} \vert {a}_{i , j} \vert , \qquad ( Eqn . 17 )$$

The sufficient graphical constraint in (Eqn. 17) implicitly requires that 1−Σ_{j∈N\{i}}|a_{i,j}|>0, which shows that the problem may not always be feasible.

By the same relationship in equation (Eqn. 10), the result is obtained.

The solutions P1G and P2G may be more computationally efficient compared to other solutions described herein. For example, there may be a single matrix (e.g., (n×n)), for P1G and a single vector (e.g., of size n) in the case of P2G that may be determined, whereas in P1C and P2C two matrices (e.g., of size (n×n)) must be found. Furthermore, P1C and P2C may have more difficult or greater constraint values than P1G and P2G. For example, P1C may have 4n² constraints and P2C may have 6n²-4n constraints, and both P1G and P2G may have n constraints.

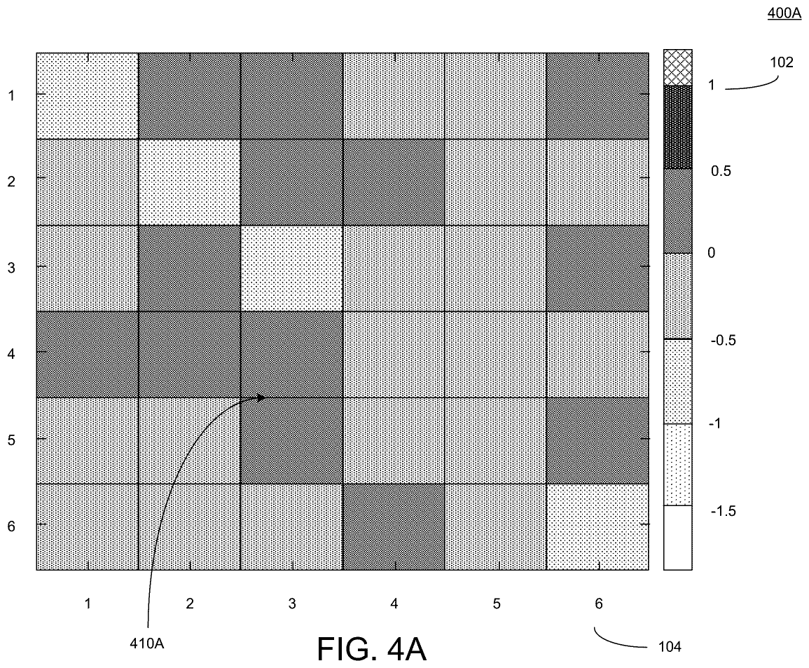

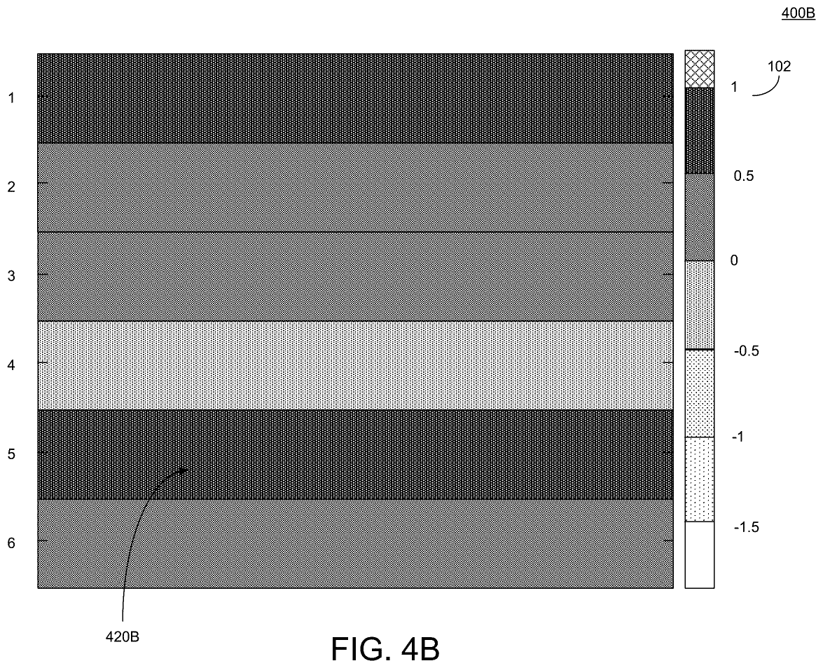

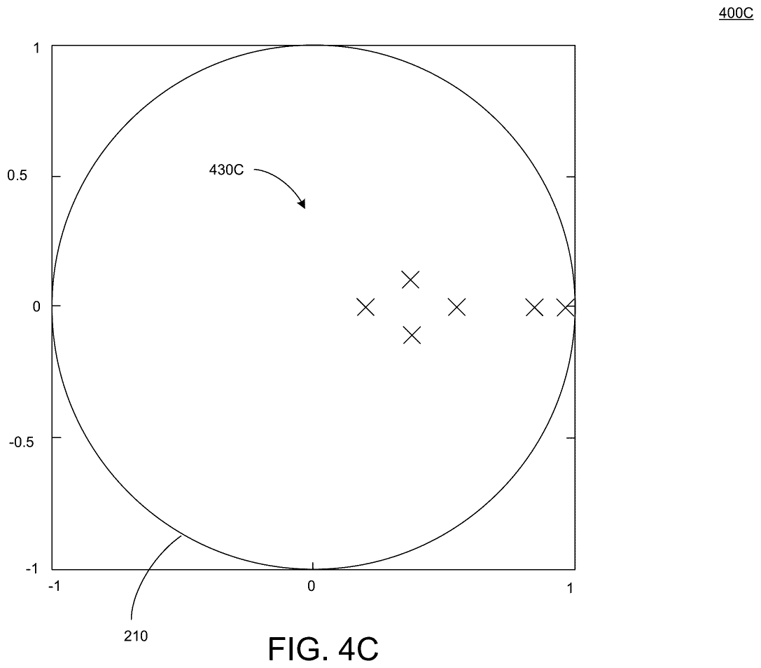

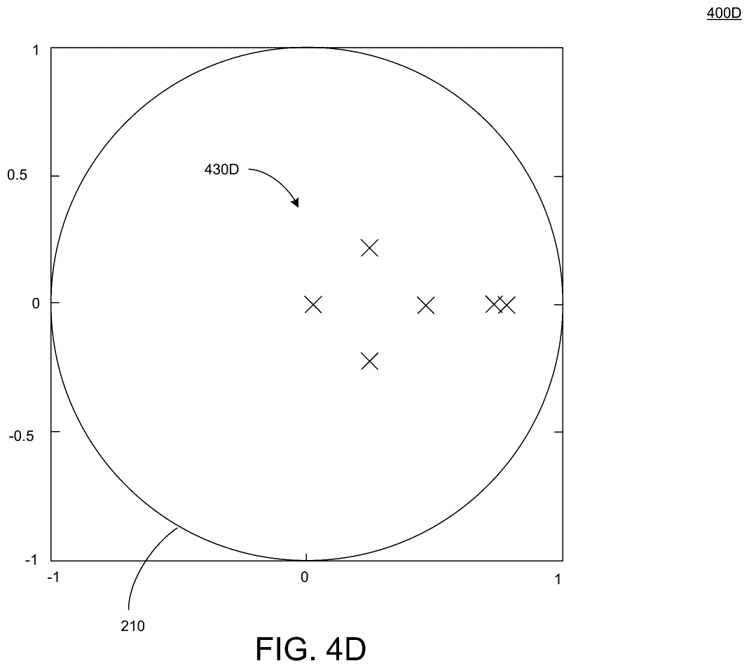

FIG. 4A depicts an example third mitigation matrix model for seizure, according to this disclosure. As illustrated by way of example in FIG. 4A, a third mitigation matrix model for seizure 400A can include at least a matrix data 410A. Specifically, the matrix 400A may be a matrix (A+Ã) indicating the graphically-interpretable sufficient approximate solution for P1C and P2C. The updated or modified spatial matrix 400A may have lower values along its diagonal relative to, for example, matrix 300A. In various embodiments, the generation of the example third mitigation matrix model of FIG. 4A may be executed by a computer system. In various embodiments still, the example third mitigation matrix model may be generated by a processor configured for or optimized for matrix and/or multivariable calculations. The matrix data 410A can indicate a stable system that is graphically-interpretable. For example, the matrix data 410A may show that for each position, the value is around zero, which indicates stability.

FIG. 4B depicts an example third mitigation exponent model for seizure, according to this disclosure. As illustrated by way of example in FIG. 4B, a third mitigation exponent model for seizure 400B can include at least an exponent data 420B. Specifically, the exponent model 400B may indicate updated, sufficient fractional order exponents (α+\tilde{α}) during seizure. The updated or modified fractional-order exponents (e.g., exponent data 420B) may have lower values compared to exponent data 320B. In various embodiments, the generation of the example third mitigation exponent model of FIG. 4B may be executed by a computer system. In various embodiments still, the example third mitigation exponent model may be generated by a processor configured for or optimized for matrix and/or multivariable calculations. The exponent data 420B can indicate that the weight of the fractional-order exponents may generally approach zero.

FIG. 4C depicts an example third mitigation eigenvalue model for seizure, according to this disclosure. As illustrated by way of example in FIG. 4C, a third mitigation eigenvalue model for seizure 400C can include at least an eigenvalues for mitigation matrix model 430C. Specifically, FIG. 4C may indicate updated eigenvalues during seizure using sufficient conditions using P1G. The updated systems may be globally asymptotically stable. In various embodiments, the generation of the example third mitigation eigenvalue model of FIG. 4C may be executed by a computer system. In various embodiments still, the example third mitigation eigenvalue model may be generated by a processor configured for or optimized for matrix and/or multivariable calculations. The eigenvalues for mitigation matrix model 430C all lie within the unit circle, indicating system stability.

FIG. 4D depicts an example third mitigation eigenvalue model for seizure, according to this disclosure. As illustrated by way of example in FIG. 4D, a third mitigation eigenvalue model for seizure 400D can include at least an eigenvalues for mitigation matrix model 430D. Specifically, FIG. 4D may indicate updated eigenvalues during seizure using sufficient conditions using P2G. In various embodiments, the generation of the example third mitigation eigenvalue model of FIG. 4D may be executed by a computer system. In various embodiments still, the example third mitigation eigenvalue model may be generated by a processor configured for or optimized for matrix and/or multivariable calculations. The eigenvalues for mitigation matrix model 430D all lie within the unit circle, indicating system stability.

In various embodiments, the matrix operations described with respect to FIGS. 1A-4D may change parameters of the complex dynamical system such that the system moves from an unstable system to a stable system. Healthcare providers, such as physicians, may utilize the generated matrices to determine epilepsy treatments for patients. In practice (e.g., when treating a patient for epilepsy), changing the system parameters could be achieved through one or more methods. For example, system parameters may be changed via target release of a drug, electrical or ultrasound neurostimulation, optogenetics, or regulation of the glia astrocytes.

In various embodiments, the generated matrices described with reference to FIGS. 1A-4D may be generated by a computer system and/or a processing system. Additionally, operations performed to generate modified, updated, or otherwise altered matrices may be performed by the computer system and/or processing system.

Having discussed specific embodiments of the present solution, it may be helpful to describe aspects of the operating environment as well as associated system components (e.g., hardware elements) in connection with the methods and systems described herein.

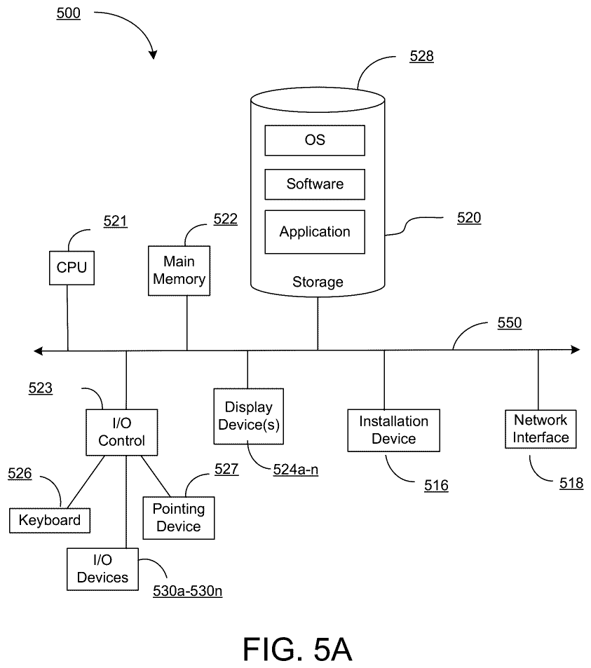

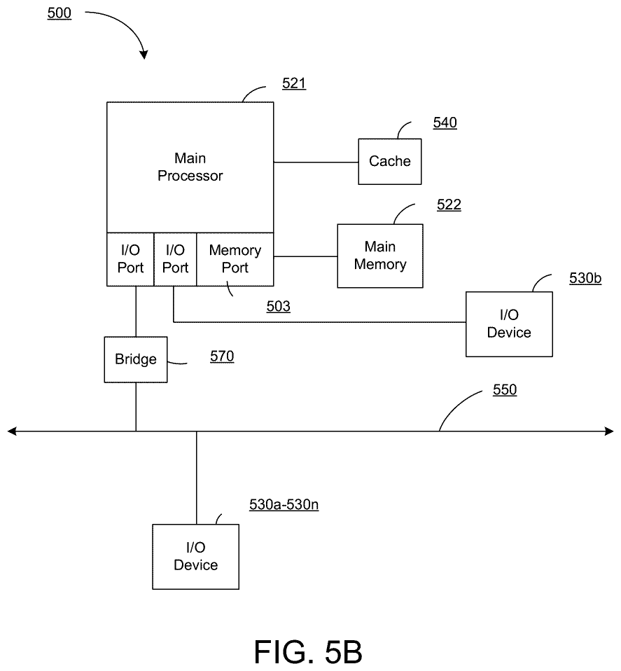

The systems discussed herein may be deployed as and/or executed on any type and form of computing device, such as a computer, network device or appliance capable of communicating on any type and form of network and performing the operations described herein. FIGS. 5A and 5B depict block diagrams of a computing device 500 useful for practicing an embodiment of the wireless communication devices 502 or the access point 506. As shown in FIGS. 5A and 5B, each computing device 500 includes a central processing unit 521, and a main memory unit 522. As shown in FIG. 5A, a computing device 500 may include a storage device 528, an installation device 516, a network interface 518, an I/O controller 523, display devices 524a-524n, a keyboard 526 and a pointing device 527, such as a mouse. The storage device 528 may include, without limitation, an operating system and/or software. As shown in FIG. 5B, each computing device 500 may also include additional optional elements, such as a memory port 503, a bridge 570, one or more input/output devices 530a-530n (generally referred to using reference numeral 530), and a cache memory 540 in communication with the central processing unit 521.

The central processing unit 521 is any logic circuitry that responds to and processes instructions fetched from the main memory unit 522. In many embodiments, the central processing unit 521 is provided by a microprocessor unit, such as: those manufactured by Intel Corporation of Mountain View, California; those manufactured by International Business Machines of White Plains, New York; or those manufactured by Advanced Micro Devices of Sunnyvale, California. The computing device 500 may be based on any of these processors, or any other processor capable of operating as described herein.

Main memory unit 522 may be one or more memory chips capable of storing data and allowing any storage location to be directly accessed by the microprocessor 521, such as any type or variant of Static random access memory (SRAM), Dynamic random access memory (DRAM), Ferroelectric RAM (FRAM), NAND Flash, NOR Flash and Solid State Drives (SSD). The main memory 522 may be based on any of the above described memory chips, or any other available memory chips capable of operating as described herein. In the embodiment shown in FIG. 5A, the processor 521 communicates with main memory 522 via a system bus 550 (described in more detail below). FIG. 5B depicts an embodiment of a computing device 500 in which the processor communicates directly with main memory 522 via a memory port 503. For example, in FIG. 5B the main memory 522 may be DRDRAM.

FIG. 5B depicts an embodiment in which the main processor 521 communicates directly with cache memory 540 via a secondary bus, sometimes referred to as a backside bus. In other embodiments, the main processor 521 communicates with cache memory 540 using the system bus 550. Cache memory 540 typically has a faster response time than main memory 522 and is provided by, for example, SRAM, BSRAM, or EDRAM. In the embodiment shown in FIG. 5B, the processor 521 communicates with various I/O devices 530 via a local system bus 550. Various buses may be used to connect the central processing unit 521 to any of the I/O devices 530, for example, a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 524, the processor 521 may use an Advanced Graphics Port (AGP) to communicate with the display 524. FIG. 5B depicts an embodiment of a computer 500 in which the main processor 521 may communicate directly with I/O device 530b, for example via HYPERTRANSPORT, RAPIDIO, or INFINIBAND communications technology. FIG. 5B also depicts an embodiment in which local busses and direct communication are mixed: the processor 521 communicates with I/O device 530a using a local interconnect bus while communicating with I/O device 530b directly.

A wide variety of I/O devices 530a-530n may be present in the computing device 500. Input devices include keyboards, mice, trackpads, trackballs, microphones, dials, touch pads, touch screen, and drawing tablets. Output devices include video displays, speakers, inkjet printers, laser printers, projectors and dye-sublimation printers. The I/O devices may be controlled by an I/O controller 523 as shown in FIG. 5A. The I/O controller may control one or more I/O devices such as a keyboard 526 and a pointing device 527, e.g., a mouse or optical pen. Furthermore, an I/O device may also provide storage and/or an installation medium 516 for the computing device 500. In still other embodiments, the computing device 500 may provide USB connections (not shown) to receive handheld USB storage devices such as the USB Flash Drive line of devices manufactured by Twintech Industry, Inc. of Los Alamitos, California.

Referring again to FIG. 5A, the computing device 500 may support any suitable installation device 516, such as a disk drive, a CD-ROM drive, a CD-R/RW drive, a DVD-ROM drive, a flash memory drive, tape drives of various formats, USB device, hard-drive, a network interface, or any other device suitable for installing software and programs. The computing device 500 may further include a storage device, such as one or more hard disk drives or redundant arrays of independent disks, for storing an operating system and other related software, and for storing application software programs such as any program or software 520 for implementing (e.g., configured and/or designed for) the systems and methods described herein. Optionally, any of the installation devices 516 could also be used as the storage device. Additionally, the operating system and the software can be run from a bootable medium.

Furthermore, the computing device 500 may include a network interface 518 to interface to the network 504 through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, IEEE 802.11ac, IEEE 802.11ad, CDMA, GSM, WiMax and direct asynchronous connections). In one embodiment, the computing device 500 communicates with other computing devices 500′ via any type and/or form of gateway or tunneling protocol such as Secure Socket Layer (SSL) or Transport Layer Security (TLS). The network interface 518 may include a built-in network adapter, network interface card, PCMCIA network card, card bus network adapter, wireless network adapter, USB network adapter, modem or any other device suitable for interfacing the computing device 500 to any type of network capable of communication and performing the operations described herein.

In some embodiments, the computing device 500 may include or be connected to one or more display devices 524a-524n. As such, any of the I/O devices 530a-530n and/or the I/O controller 523 may include any type and/or form of suitable hardware, software, or combination of hardware and software to support, enable or provide for the connection and use of the display device(s) 524a-524n by the computing device 500. For example, the computing device 500 may include any type and/or form of video adapter, video card, driver, and/or library to interface, communicate, connect or otherwise use the display device(s) 524a-524n. In one embodiment, a video adapter may include multiple connectors to interface to the display device(s) 524a-524n. In other embodiments, the computing device 500 may include multiple video adapters, with each video adapter connected to the display device(s) 524a-524n. In some embodiments, any portion of the operating system of the computing device 500 may be configured for using multiple displays 524a-524n. One ordinarily skilled in the art will recognize and appreciate the various ways and embodiments that a computing device 500 may be configured to have one or more display devices 524a-524n.

In further embodiments, an I/O device 530 may be a bridge between the system bus 550 and an external communication bus, such as a USB bus, an Apple Desktop Bus, an RS-232 serial connection, a SCSI bus, a FireWire bus, a Fire Wire 800 bus, an Ethernet bus, an AppleTalk bus, a Gigabit Ethernet bus, an Asynchronous Transfer Mode bus, a FibreChannel bus, a Serial Attached small computer system interface bus, a USB connection, or a HDMI bus.

A computing device 500 of the sort depicted in FIGS. 5A and 5B may operate under the control of an operating system, which control scheduling of tasks and access to system resources. The computing device 500 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: Android, produced by Google Inc.; WINDOWS 7 and 8, produced by Microsoft Corporation of Redmond, Washington; MAC OS, produced by Apple Computer of Cupertino, California; WebOS, produced by Research In Motion (RIM); OS/2, produced by International Business Machines of Armonk, New York; and Linux, a freely-available operating system distributed by Caldera Corp. of Salt Lake City, Utah, or any type and/or form of a Unix operating system, among others.

The computer system 500 can be any workstation, telephone, desktop computer, laptop or notebook computer, server, handheld computer, mobile telephone or other portable telecommunications device, media playing device, a gaming system, mobile computing device, or any other type and/or form of computing, telecommunications or media device that is capable of communication. The computer system 500 has sufficient processor power and memory capacity to perform the operations described herein.

In some embodiments, the computing device 500 may have different processors, operating systems, and input devices consistent with the device. For example, in one embodiment, the computing device 500 is a smart phone, mobile device, tablet or personal digital assistant. In still other embodiments, the computing device 500 is an Android-based mobile device, an iPhone smart phone manufactured by Apple Computer of Cupertino, California, or a Blackberry or WebOS-based handheld device or smart phone, such as the devices manufactured by Research In Motion Limited. Moreover, the computing device 500 can be any workstation, desktop computer, laptop or notebook computer, server, handheld computer, mobile telephone, any other computer, or other form of computing or telecommunications device that is capable of communication and that has sufficient processor power and memory capacity to perform the operations described herein.

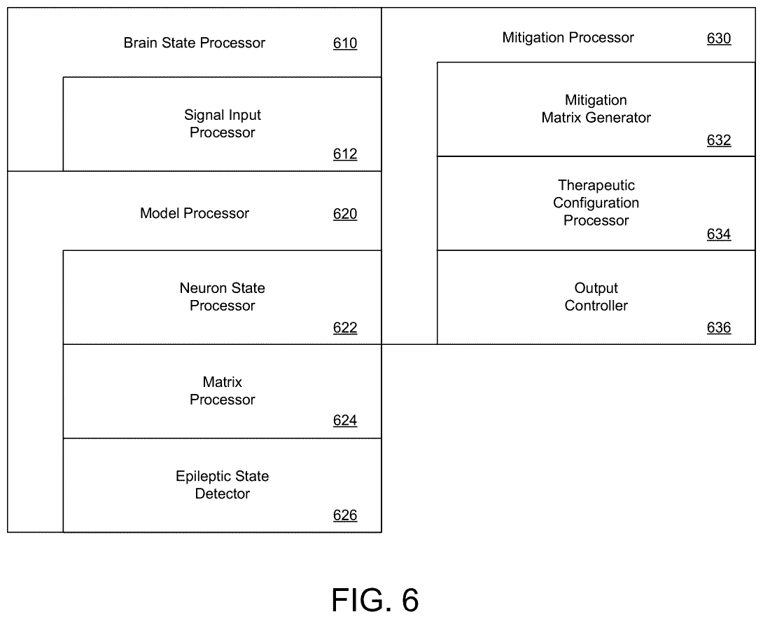

FIG. 6 depicts an example computer architecture, according to this disclosure. As illustrated by way of example in FIG. 6, a computer architecture 600 can include at least a brain state processor 610, a model processor 620, and a mitigation processor 630. The one or more processors may be constructed in a manner sufficient to perform at least the operations described herein. For example, one or more of the brain state processor 610, the model processor 620, and the mitigation processor 630 may execute instructions stored in memory (e.g., a memory similar to the main memory 522) or may execute instructions otherwise accessible to the processors. Alternatively or additionally, the processor(s) may be implemented as hardware, firmware, software, operating systems, embedded operating systems, or the like.

The brain state processor 610 can determine a brain state of a patient. For example, the brain state processor 610 may determine a level or type of brain activity of a patient and corresponding information about the patient. For example, the brain state processor 610 may determine whether a patient is awake, in a REM cycle, lacking brain function, experiencing improper brain function, or the like. The brain state processor 610 can include a signal input processor 612.

The signal input processor 612 can receive a signal output by the brain of a patient and determine brain activity associated with the signal. The signal from the brain may be an input to the computer architecture 600 for use in generating a plurality of matrices corresponding to brain activity indicative of an event of the brain corresponding to epilepsy. For example, the signal input processor 612 may receive one or more brain waves. The signal input processor 612 may utilize the brain waves to determine brain function and/or a brain state. The data collected by the signal input processor 612 may be used in generation of a model corresponding to a state of one or more neurons of the brain of the patient.

The model processor 620 can generate one or more models corresponding to a state of a network of neurons. For example, the model processor 620 may utilize information about the network of neurons from the signal input processor 612 to generate a model (e.g., a computer model or the like) of the network of neurons. The model may include, for example, information about activity, actions, or the like, of individual neurons in the network of neurons. The model may also include a representation of how the neurons interact with one another in various brain states. For example, the model may indicate how neurons behave and interact when the patient is experiencing a seizure. The model processor 620 can include a neuron state processor 622, a matrix processor 624, and an epileptic state detector 626.

The neuron state processor 622 can determine a state of one or more neurons of the network. For example, the neuron state processor 622 may receive information on neuron activity, via, for example, one or more sensors configured to monitor brain activity. The neuron activity may be, in some embodiments, specific to the network of neurons (e.g., an area of interest of the brain). The neuron state processor 622 may receive sensor data indicating the state of one or more neurons. A state of a neuron may indicate what actions the neuron is performing at a specific point in time. For example, a neuron may be in a firing state (e.g., an action potential is occurring) or a resting state (e.g., no action potential is occurring).

The matrix processor 624 can generate a first matrix indicative of activity of the network of neurons. The matrix processor 624 may receive information relating to the state of one or more neurons (e.g., from the neuron state processor 622) to determine whether a matrix should be generated and to generate the matrix. The matrix processor 624 may also receive the model generated by the model processor 620 and use the model to generate the matrix. For example, responsive to receiving the neuron state data, the matrix processor 624 may determine that neurons are firing in such a way that indicates a seizure or other event relating to epilepsy is occurring in the patient's brain. Additionally, the model may indicate a number of neurons, interactions between neurons, or the like. as inputs for the generation of the first matrix. Using the received data, the matrix processor 614 may generate a first matrix. The first matrix may be indicative of the patient's brain before a seizure. For example, the first matrix may be similar to the matrix 100A of FIG. 1A. In various embodiments, the first matrix may be indicative of the patient's brain during a seizure. For example, the first matrix may be similar to the matrix 300A of FIG. 3A. The matrix processor 624 may utilize the discrete-time linear fractional-order systems described with respect to FIGS. 1A-4D to generate a matrix corresponding to neural activity of the patient.

In various embodiments, the matrix processor 624 may also generate one or more graphs showing fractional-order exponents corresponding to the generated matrices. For example, when the matrix processor 624 generates the matrix 100A, the matrix processor 624 may also generate a corresponding exponent model 100B of FIG. 1B. The matrix processor 624 may also perform various operations to generate an eigenvalue model corresponding to the generated matrix and/or exponent model. For example, upon generating the matrix 100A, the matrix processor 624 may generate the corresponding eigenvalue model 200A. Upon generation of the first matrix, the matrix processor 624 may determine whether the matrix indicates a stable or unstable system. For example, when the first matrix is generated based on neuron data indicative of a seizure, the matrix processor 624 may determine that the model system is unstable.

In various embodiments, the matrix processor 624 may generate a second matrix corresponding to an altered or modified matrix (e.g., matrix 300C of FIG. 3C). The altered or modified matrix may be determined to be indicative of an unstable system. For example, the matrix processor 624 may perform matrix operations on the first matrix to satisfy P1 and generate the altered or modified second matrix. Further, the matrix processor 624 may generate a corresponding altered exponent model to satisfy P2. For example, the matrix processor 624 may generate the matrix 300C as the second matrix and may generate the altered or modified exponent model 300D of FIG. 3D.

The epileptic state detector 626 can detect an epileptic event of a patient and/or a state of an epileptic event of the patient. For example, the epileptic state detector 626 may receive information relating to neural activity (e.g., from the neuron state processor 622). The epileptic state detector 626 may also receive information from the matrix processor 624. Specifically, the epileptic state detector 626 may receive an eigenvalue model generated by the matrix processor 624. The epileptic state detector 626 may utilize the eigenvalue model to determine that one or more eigenvalues satisfy a condition indicative of the activity during the event of the brain corresponding to epilepsy. For example, the epileptic state detector 626 may determine that the eigenvalue model includes unstable eigenvalues (e.g., unstable eigenvalues 224A), indicating that the patient may experience a seizure. In various embodiments, the condition may correspond to a predetermined number of one or more positions of one or more of the eigenvalues outside a unit circle based on the first matrix. The unit circle may be indicative of a state of stability or instability of the brain with respect to epilepsy.

The mitigation processor 630 can perform operations to mitigate instability of the system and determine, using the model, treatment to deliver to the patient to treat epilepsy and/or a particular seizure (e.g., an ongoing seizure). For example, the mitigation processor 630 may receive the first matrix generated by the matrix processor 624. The mitigation processor 630 may analyze the first matrix and corresponding data to determine matrix operations to perform to stabilize the system (e.g., stabilize the system representative of the brain or network of neurons of the patient). Responsive to stabilizing the representative system based on the model generated by the model processor 620, the mitigation processor 630 may determine corresponding actions or treatments for a health care provider to take to stabilize the system of the patient (e.g., the brain of the patient) in a manner corresponding to the stabilization of the model system. For example, the matrix operations performed to generate a second matrix indicating a stable system may correspond to operations to take (e.g., stimulation, drug delivery) to treat, prevent, or mitigate a seizure or epilepsy in a patient. The mitigation processor 630 can include a mitigation matrix generator 632, a therapeutic configuration controller 634, and an output controller 636.

The mitigation matrix generator 632 can generate one or more matrices corresponding to a stable system. The mitigation matrix generator 632 may generate a third matrix based on the first matrix and/or second matrices (e.g., the spatial matrix 100A and/or the altered or modified spatial matrix 300C) generated by the matrix processor 624. In various embodiments, the mitigation matrix generator 632 may generate the second matrix (e.g., the altered matrix) instead of the matrix processor 620. The mitigation matrix generator 632 may modify the first matrix to achieve system stability (e.g., global asymptotic stability). The mitigation matrix generator 632 may also perform convexification of P1 and P2.

Responsive to convexification of P1 and P2, the mitigation matrix generator 632 may generate a third matrix. The third matrix may be an updated or modified spatial matrix (e.g., matrix 300E of FIG. 3E). The updated or modified spatial matrix may be generated responsive to or as a solution of P1C. Additionally, the mitigation matrix generator 632 may generate an updated or modified exponent model (e.g., exponent model 300F of FIG. 3F) responsive to or as a solution of P2C. The third matrix may indicate system stability. For example, the matrix operations performed on the first matrix to arrive at the third matrix may indicate operations that cause the system to become stable from instability.

In various embodiments, the mitigation matrix generator 632 may generate a fourth matrix. The fourth matrix may be an updated or modified sufficient spatial matrix (e.g., the matrix 400A of FIG. 4A). The updated or modified sufficient spatial matrix may be generated as a less complex and/or graphically-interpretable solution to P1C. The fourth matrix may also indicate system stability. The system stability may correspond to or indicate mitigation of the event corresponding to epilepsy in the patient (e.g., a seizure). Additionally, the mitigation matrix generator 632 may generate an updated or modified exponent model (e.g., exponent model 400B of FIG. 4B) responsive to or as a solution of P2C. The mitigation matrix generator 632 may also generate one or more eigenvalue models corresponding to matrices indicating system stability. For example, the mitigation matrix generator 632 may generate eigenvalue models 400C and/or 400D corresponding to P1G and P2G, respectively.

In various embodiments, based on one or more eigenvalues corresponding to the second matrix, the mitigation matrix generator 632 may determine that the second matrix is indicative of mitigation of the activity during the event of the brain corresponding to epilepsy. Additionally, the mitigation matrix generator 632 determine that the one or more eigenvalues generated in the eigenvalue model (e.g., eigenvalue model 400C), satisfy a condition indicative of the mitigation of activity during the event of the brain corresponding to epilepsy. In various embodiments, the condition may correspond to a predetermined number of one or more positions of one or more of the eigenvalues inside a unit circle based on the first matrix. The unit circle may be indicative of a state of stability or instability of the brain with respect to epilepsy.

The mitigation matrix generator 632 may generate any combination of the second, third, and/or fourth matrices and any corresponding exponent models and/or eigenvalue models. For example, upon receiving the first matrix, the mitigation matrix generator 632 may perform multiple operations to arrive at the updated or modified sufficient matrix without producing the altered matrix or the updated matrix.

The therapeutic configuration controller 634 can determine treatment to deliver to a patient to treat epilepsy and/or a specific seizure. The treatment determined by the therapeutic configuration controller 634 may be based on or correspond to the matrix generated by the mitigation matrix generator 632. For example, the therapeutic configuration controller 634 may receive the stable matrix and/or information corresponding to the stable matrix generated by the mitigation matrix generator 632. The therapeutic configuration controller 634 may identify matrix operations performed that cause a first matrix indicating system instability (e.g., matrix 100A or matrix 300C) to be modified to a second matrix indicating system stability (e.g., matrix 300E or matrix 400A). The therapeutic configuration controller 634 may determine actions or treatments corresponding to the matrix operations that may treat, mitigate, and/or prevent the brain event corresponding to epilepsy (e.g., a seizure). For example, the unstable system indicated by the first matrix may represent a brain event corresponding to epilepsy in a patient (e.g., a seizure). Upon performing one or more matrix operations as discussed herein, the second matrix may indicate a stable system that may represent mitigation of the brain event corresponding to epilepsy. In order to realize the mitigation of the brain event indicated by the second matrix, the therapeutic configuration controller 634 may determine treatment to achieve the mitigation. The treatment may have a physical property based on the first matrix and the second matrix. The physical property may be, for example, a chemical, an electrical signal, an ultrasound signal, electromagnetic radiation, or a biochemical. In various embodiments, the treatment may be a therapeutic response corresponding to release of a drug to the brain of the patient, stimulation of the brain of the patient, an optogenetic treatment to the brain of the patient, or application of glia astrocytes to the patient. The therapeutic configuration controller 634 may determine, for example, that stimulation should be delivered to the patient at a specific frequency and/or amplitude in order to achieve the same results as modeled or otherwise indicated by the updated or modified matrix.

The output controller 636 can control and/or modulate a delivery of the treatment determined by the therapeutic configuration controller 634. For example, the output controller 636 may receive, from the therapeutic configuration controller 634, information relating to a treatment of the brain event corresponding to epilepsy. The output controller 636 may control operation of a device configured to deliver the treatment. For example, the output controller 636 may modulate a stimulation to a nerve or neuron that is meant to treat the brain event corresponding to epilepsy to provide proper stimulation (e.g., for a proper duration, intensity, dosage, or the like).

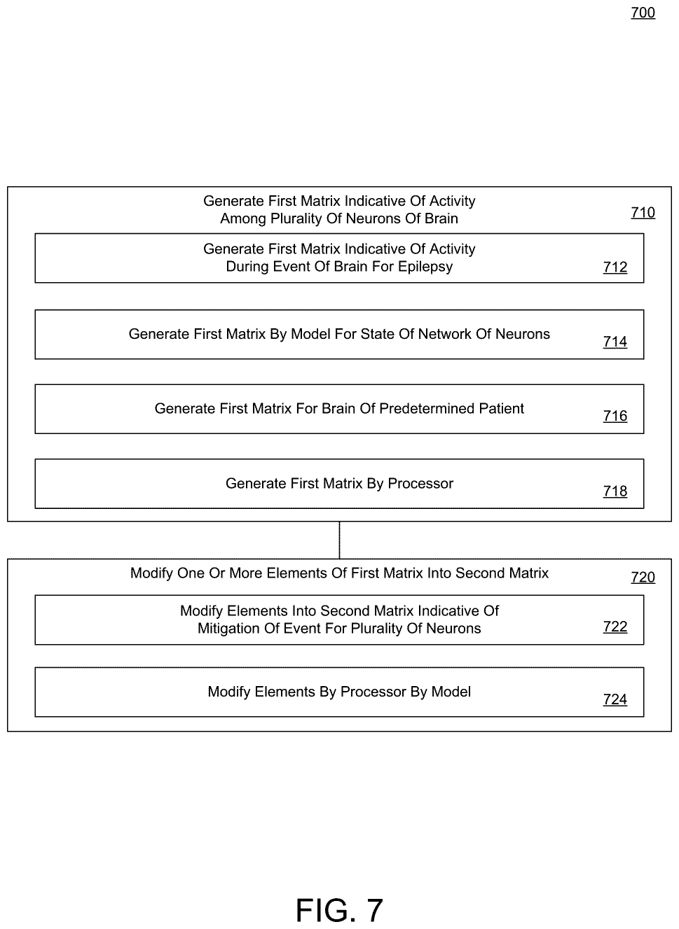

FIG. 7 depicts an example method of detection and mitigation of brain event, according to this disclosure. At least one or more components of the computer architecture 600 can perform method 700. For example, the model processor 620, and/or the mitigation processor 630 may perform the method 700.

At 710, the method 700 can generate a first matrix indicative of activity among a plurality of neurons of a brain. At 712, the method 700 can generate a first matrix indicative of activity during an event of the brain for epilepsy. For example, the model processor 620 may determine that a patient is experiencing an epileptic event (e.g., a seizure). The generated first matrix may indicate an unstable system corresponding to the epileptic event. At 714, the method 700 can generate the first matrix according to a model for a state of a network of neurons. At 716, the method 700 can generate the first matrix for a brain of a predetermined patient. For example, a predetermined patient may receive epilepsy treatment according to the methods described herein. At 718, the method 700 can generate the first matrix by a processor. In an aspect, the method can include determining, based on one or more eigenvalues corresponding to the first matrix, that the first matrix is indicative of the activity during the event of the brain corresponding to epilepsy. For example, the model processor 620 may generate an eigenvalue model corresponding to the first matrix. The eigenvalue model include a plurality of eigenvalues located within, on, and beyond a unit circle. In an aspect, the method 700 can include determining, by the processor, that the one or more eigenvalues satisfy a condition indicative of the activity during the event of the brain corresponding to epilepsy. In an aspect, the condition may correspond to a predetermined number of one or more positions of one or more of the eigenvalues outside the unit circle based on the first matrix. and where the unit circle is indicative of a state of stability of the brain with respect to epilepsy. For example, when one or more eigenvalues are positioned beyond the unit circle, the system may be considered unstable, which may correspond to brain activity being indicative of an epileptic event

At 720, the method 700 can modify one or more elements of the first matrix into a second matrix. At 722, the method 700 can modify the elements into a second matrix indicative of mitigation of the event for the plurality of neurons. At 724, the method 700 can modify the elements by the processor according to the model. In an aspect, the method can include determining, by the processor and based on one or more eigenvalues corresponding to the second matrix, that the second matrix is indicative of mitigation of the activity during the event of the brain corresponding to epilepsy. In an aspect, the method can include determining, by the processor, that the one or more eigenvalues satisfy a condition indicative of the mitigation of the activity during the event of the brain corresponding to epilepsy. For example, the unit circle may be indicative of a state of stability of the brain with respect to epilepsy. Additionally, the condition may correspond to a predetermined number of one or more positions of one or more of the eigenvalues inside a unit circle based on the second matrix. For example, a generated eigenvalue model may include one or more eigenvalues located within the unit circle. When no eigenvalues exist on or outside of the unit circle, the eigenvalue model may indicate a stable system. A stable system may correspond to mitigation of the epileptic event.

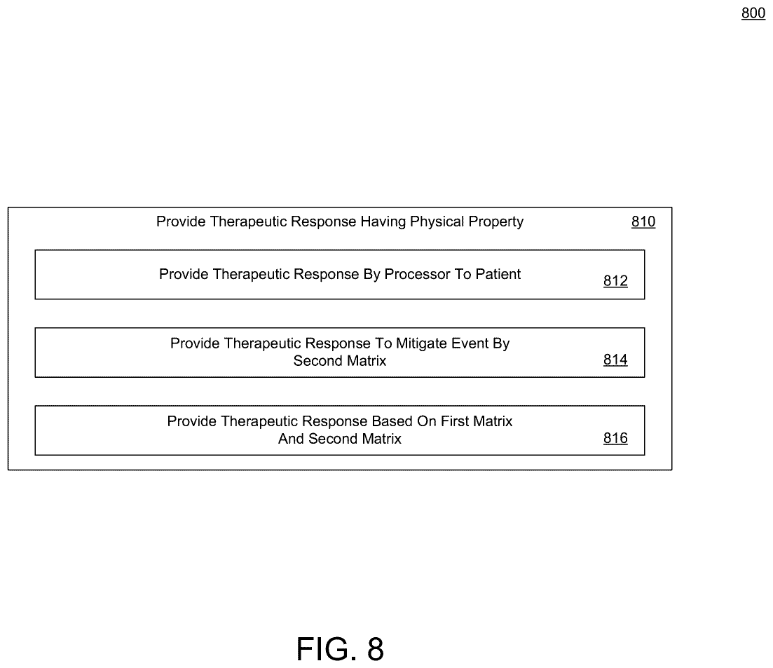

FIG. 8 depicts an example method of detection and mitigation of brain event, according to this disclosure. At least one or more components of the computer architecture 600 can perform method 800. For example, the model processor 620, and/or the mitigation processor 630 may perform the method 800.

At 810, the method 800 can provide a therapeutic response having a physical property. At 812, the method 800 can provide the therapeutic response by the processor to the patient. At 814, the method 800 can provide the therapeutic response to mitigate the event according to the second matrix. At 816, the method 800 can provide the therapeutic response based on the first matrix and the second matrix. In an aspect, the method 800 can include where the therapeutic response corresponds to release of a drug to the brain of the patient, stimulation of the brain of the patient, an optogenetic treatment to the brain of the patient, or application of glia astrocytes to the patient. In an aspect, the method 800 can include where the physical property corresponds to at least one of a chemical, an electrical signal, an ultrasound signal, electromagnetic radiation, or a biochemical.

The various illustrative logical blocks, modules, circuits, and algorithm steps described in connection with the embodiments disclosed herein may be implemented as electronic hardware, computer software, or combinations of both. To illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, or the like may be passed, forwarded, or transmitted via any suitable means including memory sharing, message passing, token passing, network transmission, or the like.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise random-access memory (RAM), read-only memory (ROM), electronically erasable programmable read-only memory (EEPROM), CD-ROM or other optical disk storage, flash storage, or other solid-state storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The operations described in this specification can be implemented as operations performed by a data processing apparatus on data stored on one or more computer-readable storage devices or received from other sources.

The terms “data processing apparatus”, “data processing system”, “client device”, “computing platform”, “computing device”, “computing system”, “user device”, or “device” can encompass all kinds of apparatus, devices, and machines for processing data, including by way of example a programmable processor, a computer, a system on a chip, or multiple ones, or combinations, of the foregoing. The apparatus can include special purpose logic circuitry, e.g., a field-programmable gate array (FPGA) or an application-specific integrated circuit (ASIC). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

Processors suitable for the execution of a computer program include, by way of example, both general and special purpose microprocessors, and any one or more processors of any kind of digital computer. Generally, a processor will receive instructions and data from a read-only memory or a random access memory or both. The elements of a computer include a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a GPS receiver, a digital camera device, a video camera device, or a portable storage device (e.g., a universal serial bus (USB) flash drive), for example. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube), plasma, or LCD monitor, for displaying information to the user; a keyboard; and a pointing device, e.g., a mouse, a trackball, or a touchscreen, by which the user can provide input to the computer. Other kinds of devices can be used to provide for interaction with a user as well; for example, feedback provided to the user can include any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user.

In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. For example, the computing devices described herein can each be a single module, a logic device having one or more processing modules, one or more servers, or an embedded computing device.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed only in connection with one implementation are not intended to be excluded from a similar role in other implementations or implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting.

What is claimed is:

1. A system, comprising:
one or more processors, coupled with memory, to:
generate, according to a model corresponding to a state of a network of neurons of a brain of a predetermined patient, a first matrix indicative of activity among a plurality of neurons of the brain during an event of the brain corresponding to epilepsy;
modify, according to the model, one or more elements of the first matrix into a second matrix indicative of mitigation of the event for the plurality of neurons; and
provide, to the patient to mitigate the event according to the second matrix, a therapeutic response having a physical property based on the first matrix and the second matrix.

2. The system of claim 1, comprising the one or more processors to:
determine, based on one or more eigenvalues corresponding to the first matrix, that the first matrix is indicative of the activity during the event of the brain corresponding to epilepsy.

3. The system of claim 2, comprising the one or more processors to:
determine that the one or more eigenvalues satisfy a condition indicative of the activity during the event of the brain corresponding to epilepsy.

4. The system of claim 3, wherein the condition corresponds to a predetermined number of one or more positions of one or more of the eigenvalues outside a unit circle based on the first matrix, and wherein the unit circle is indicative of a state of stability of the brain with respect to epilepsy.

5. The system of claim 1, comprising the one or more processors to:
determine, based on one or more eigenvalues corresponding to the second matrix, that the second matrix is indicative of mitigation of the activity during the event of the brain corresponding to epilepsy.

6. The system of claim 2, comprising the one or more processors to:
determine that the one or more eigenvalues satisfy a condition indicative of the mitigation of the activity during the event of the brain corresponding to epilepsy.

7. The system of claim 3, wherein the condition corresponds to a predetermined number of one or more positions of one or more of the eigenvalues inside a unit circle based on the first matrix, and wherein the unit circle is indicative of a state of stability of the brain with respect to epilepsy.

8. The system of claim 1, wherein the therapeutic response corresponds to release of a drug to the brain of the patient, stimulation of the brain of the patient, an optogenetic treatment to the brain of the patient, or application of glia astrocytes to the patient.

9. The system of claim 1, wherein the physical property corresponds to at least one of a chemical, an electrical signal, an ultrasound signal, electromagnetic radiation, or a biochemical.

10. A method, comprising:
generating, by a processor according to a model corresponding to a state of a network of neurons of a brain of a predetermined patient, a first matrix indicative of activity among a plurality of neurons of the brain during an event of the brain corresponding to epilepsy;
modifying, by the processor according to the model, one or more elements of the first matrix into a second matrix indicative of mitigation of the event for the plurality of neurons; and
providing, by the processor to the patient to mitigate the event according to the second matrix, a therapeutic response having a physical property based on the first matrix and the second matrix.

11. The method of claim 10, further comprising:
determining, based on one or more eigenvalues corresponding to the first matrix, that the first matrix is indicative of the activity during the event of the brain corresponding to epilepsy.

12. The method of claim 11, further comprising:
determining, by the processor, that the one or more eigenvalues satisfy a condition indicative of the activity during the event of the brain corresponding to epilepsy.

13. The method of claim 12, wherein the condition corresponds to a predetermined number of one or more positions of one or more of the eigenvalues outside a unit circle based on the first matrix, and wherein the unit circle is indicative of a state of stability of the brain with respect to epilepsy.

14. The method of claim 10, further comprising:
determining, by the processor and based on one or more eigenvalues corresponding to the second matrix, that the second matrix is indicative of mitigation of the activity during the event of the brain corresponding to epilepsy.

15. The method of claim 11, further comprising:
determining, by the processor, that the one or more eigenvalues satisfy a condition indicative of the mitigation of the activity during the event of the brain corresponding to epilepsy.

16. The method of claim 15, wherein the condition corresponds to a predetermined number of one or more positions of one or more of the eigenvalues inside a unit circle based on the first matrix, and wherein the unit circle is indicative of a state of stability of the brain with respect to epilepsy.

17. The method of claim 10, wherein the therapeutic response corresponds to release of a drug to the brain of the patient, stimulation of the brain of the patient, an optogenetic treatment to the brain of the patient, or application of glia astrocytes to the patient.

18. The method of claim 10, wherein the physical property corresponds to at least one of a chemical, an electrical signal, an ultrasound signal, electromagnetic radiation, or a biochemical.

19. A non-transitory computer readable medium including one or more instructions stored thereon and executable by a processor to:
generate, by a processor according to a model corresponding to a state of a network of neurons of a brain of a predetermined patient, a first matrix indicative of activity among a plurality of neurons of the brain during an event of the brain corresponding to epilepsy;
modify, by the processor according to the model, one or more elements of the first matrix into a second matrix indicative of mitigation of the event for the plurality of neurons; and
provide, by the processor to the patient to mitigate the event according to the second matrix, a therapeutic response having a physical property based on the first matrix and the second matrix.

20. The non-transitory computer readable medium of claim 19, wherein the therapeutic response corresponds to release of a drug to the brain of the patient, stimulation of the brain of the patient, an optogenetic treatment to the brain of the patient, or application of glia astrocytes to the patient.
